(19) United States
(12) Patent Application Publication
(10) Pub. No.: US 2026/0139037 A1
(43) Pub. Date: May 21, 2026

(54) COMPOSITIONS AND METHODS FOR HIV LATENCY REVERSAL

(71) Applicant: The Trustees of Columbia University in the City of New York, New York, NY (US)

(72) Inventors: David D. HO, Bedford, NY (US); Yaoxing HUANG, Brooklyn, NY (US); Jian YU, New Providence, NJ (US); Hiroshi MOHRI, New York, NY (US); Manoj S. NAIR, Fair Lawn, NJ (US)

(21) Appl. No.: 19/327,506

(22) Filed: Sep. 12, 2025

Related U.S. Application Data
(63) Continuation-in-part of application No. PCT/US24/19815, filed on Mar. 13, 2024.
(60) Provisional application No. 63/489,982, filed on Mar. 13, 2023.

(51) Int. Cl. C07K 16/114; A61P 37/04; C07K 14/54

(52) U.S. Cl. CPC C07K 16/1145; A61P 37/04; C07K 14/5443; C07K 2317/52; C07K 2319/00

(57) ABSTRACT

In some embodiments, the subject matter described herein relates to a bispecific molecule for the treatment of HIV-1 latent infections, the molecule comprising an anti-CD4 iMab portion and an IL-15/IL-15Rα Fc portion.

This application is a continuation-in-part of International Application No. PCT/US2024/019815, filed on Mar. 13, 2024, which claims priority to U.S. Provisional Patent Application No. 63/489,982, filed on Mar. 13, 2023, the contents of which are hereby incorporated by reference in their entireties.

The instant application contains a Sequence Listing which has been submitted electronically in XML file format and is hereby incorporated by reference in its entirety. Said XML copy, created on Sep. 12, 2025, is named 0019240_01309US2_SL.xml and is 15,600 bytes in size.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosure of these publications in their entireties are hereby incorporated by reference into this application in order to more fully describe the state of the art as known to those skilled therein as of the date of the invention described herein.

### BACKGROUND

Human immunodeficiency virus (HIV) is a virus which attacks the human immune system. If the HIV infection is not treated, it can lead to the development of an acquired immunodeficiency syndrome (AIDS). Currently, there is no effective cure for HIV, however, with proper medical care, the infection can be controlled.

### SUMMARY OF THE INVENTION

In certain aspects, the subject matter described herein provides a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide and a fourth polypeptide, wherein the third polypeptide comprises CH2—CH3 domains of a heavy chain and the fourth polypeptide comprises an IL-15 domain, wherein the CH2-CH3 domains of the heavy chain are coupled to IL-15 domain; and wherein the heavy chain of the first arm and the Ch2-CH3 domains of the second arm form a knob-into-hole structure.

In some aspects, the subject matter described herein provides a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide, wherein the third polypeptide comprises CH2-CH3 domains of a heavy chain, wherein the second arm further comprises an IL-15 domain; and wherein the heavy chain domain of the first arm and the CH2-CH3 domains of the second arm form a knob-into-hole structure.

In some embodiments, the first arm forms a first antigen binding region. In some embodiments, the first arm binds CD4. In some embodiments, the first arm binds CD4 with a first antigen binding region. In some embodiments, the second arm forms a second antigen binding region. In some embodiments, the second arm binds an IL-15 receptor. In some embodiments, the second arm binds an IL-15 receptor with the second binding region. In some embodiments, the heavy chain comprises an IL-15 domain and CH2-CH3 constant domains, wherein the CH2 domain is linked to the IL-15 domain via a GSx3 linker. In some embodiments, the heavy chain comprises an IL-15 receptor-α domain and CH2-CH3 constant domains, wherein the CH2 domain is linked to the IL-15 receptor-α domain via a GSx3 linker. In some embodiments, the CH2 domain is linked to the IL-15 receptor-α domain at a sushi domain. In some embodiments, the CH2-CH3 domains are the IgG1 Fc CH2-CH3 domains. In some embodiments, the IgG Fc domain comprises LALA mutations. LALA mutations can refer to L234A/L235A mutations in the IgG Fc domain. In some embodiments, the IgG Fc domain comprises a triple mutation (TM). TM can refer to L234F/L235E/P329S mutations in the IgG Fc domain. In some embodiments, the heavy chain of the first arm is coupled to the light chain of the first arm via one or more disulfide bonds. In some embodiments, the CH2-CH3 domains of the second arm are coupled to an IL-15 domain via a GSx3 linker. In some embodiments, the heavy chain of the first arm is coupled the CH2-CH3 domain of the second arm via one or more disulfide bonds. In some embodiments, the bispecific molecule further comprises a Sushi domain of an IL-15 receptor α chain. In some embodiments, the Sushi domain is coupled to the CH2-CH3 domains of the second arm via a GSx3 linker. In some embodiments, the Sushi domain is coupled to the IL-15 domain via one or more disulfide bonds. In some embodiments, the CH2-CH3 domains of the second arm are not directly coupled to the IL-15 domain.

In some embodiments, the light chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the light chain of the first arm comprises an amino acid sequence with SEQ ID NO: 1. In some embodiments, the light chain of the first arm comprises a variable (VL) domain and a constant (CL) domain, wherein the VL comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 9. In some embodiments, the light chain of the first arm comprises a variable (VL) domain and a constant (CL) domain, wherein the VL comprises an amino acid sequence with SEQ ID NO: 9. In some embodiments, the heavy chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 2. In some embodiments, the heavy chain of the first arm comprises an amino acid sequence with SEQ ID NO: 2. In some embodiments, the second arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 3. In some embodiments, the second arm comprises an amino acid sequence with SEQ ID NO: 3. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 4. In some embodiments, the IL-15 domain comprises an amino acid sequence with SEQ ID NO: 4. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 10. In some embodiments, the IL-15 domain comprises an amino acid sequence with SEQ ID NO: 10.

In some embodiments, the bispecific molecule is conjugated to a histone deacetylases (HDAC) inhibitor. In some embodiments, the HDAC inhibitor is Romidepsin (RMD). In some embodiments, the bispecific molecule is conjugated to a PKC activator. In some embodiments, the PKC activator is Ingenol mebutate. In some embodiments, the bispecific molecule is conjugated to a PTEN inhibitor. In some embodiments, the PTEN inhibitor is Disulfiram. In some embodiments, the bispecific molecule is conjugated to a RIG-1 activator. In some embodiments, the RIG-1 activator is Acitretin. In some embodiments, the bispecific molecule is conjugated to a SMAC mimetic. In some embodiments, the SMAC mimetic is Birinapant. In some embodiments, the bispecific molecule is T4IL15. In some embodiments, the bispecific molecule is T4IL15(-Rα).

In certain aspects, the subject matter described herein provides a pharmaceutical composition comprising any of the bispecific molecule disclosed herein.

In certain aspects, the subject matter described herein provides a polynucleotide encoding any of the bispecific molecule disclosed herein or a fragment thereof.

In certain aspects, the subject matter described herein provides a virus comprising a polynucleotide encoding any of the bispecific molecule disclosed herein or a fragment thereof.

In certain aspects, the subject matter described herein provides a genetically engineered cell comprising any of the bispecific molecule disclosed herein.

In certain aspects, the subject matter described herein provides a genetically engineered cell comprising any of the bispecific molecule disclosed herein or a fragment thereof.

In certain aspects, the subject matter described herein provides a method of treating latent HIV-1 infection in a subject in need thereof, the method comprising administering to the subject a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide and a fourth polypeptide, wherein the third polypeptide comprises CH2-CH3 domains of a heavy chain and the fourth polypeptide comprises an IL-15 domain, wherein the CH2-CH3 domains of the heavy chain are coupled to IL-15 domain; and wherein the heavy chain of the first arm and the Ch2-CH3 domains of the second arm form a knob-into-hole structure.

In certain aspects, the subject matter described herein provides a method of treating latent HIV-1 infection or reactivating a HIV-1 reservoir in a subject in need thereof, the method comprising administering to the subject a bispecific molecule comprising: a first arm comprising a first polypeptide and a second polypeptide, wherein the first polypeptide comprises a heavy chain domain and the second polypeptide comprises a light chain, wherein the heavy chain is coupled to the light chain, a second arm comprising a third polypeptide, wherein the third polypeptide comprises CH2-CH3 domains of a heavy chain, wherein the second arm further comprises an IL-15 domain; and wherein the heavy chain of the first arm and the CH2-CH3 domains of the second arm form a knob-into-hole structure.

In some embodiments, the first arm forms a first antigen binding region. In some embodiments, the first antigen binding region binds CD4. In some embodiments, the second arm forms a second antigen binding region. In some embodiments, the second antigen binding region binds an IL-15 receptor. In some embodiments, the heavy chain comprises CH1-CH3 constant domains and a variable domain, wherein the CH2 domain is linked to the CH1 via a GSx3 linker. In some embodiments, the CH2-CH3 domains are the IgG1 Fc CH2-CH3 domains. In some embodiments, the IgG Fc domain comprises LALA mutations. In some embodiments, the heavy chain of the first arm is coupled to the light chain of the first arm via one or more disulfide bonds. In some embodiments, the CH2-CH3 domains of the second arm are coupled to an IL-15 domain via a GSx3 linker. In some embodiments, the heavy chain of the first arm is coupled to the CH2-CH3 domain of the second arm via one or more disulfide bonds. In some embodiments, the bispecific molecule further comprises a Sushi domain of an IL-15 receptor α chain. In some embodiments, the Sushi domain is coupled to the CH2-CH3 domains of the second arm via a GSx3 linker. In some embodiments, the Sushi domain is coupled to the IL-15 domain via one or more disulfide bonds. In some embodiments, the CH2-CH3 domains of the second arm are not directly coupled to the IL-15 domain.

In some embodiments, the light chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the light chain of the first arm comprises an amino acid sequence with SEQ ID NO: 1. In some embodiments, the light chain of the first arm comprises a variable (VL) domain and a constant (CL) domain, wherein the VL comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 9. In some embodiments, the light chain of the first arm comprises a variable (VL) domain and a constant (CL) domain, wherein the VL comprises an amino acid sequence with SEQ ID NO: 9. In some embodiments, the heavy chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 2. In some embodiments, the heavy chain of the first arm comprises an amino acid sequence with SEQ ID NO: 2. In some embodiments, the second arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 3. In some embodiments, the second arm comprises an amino acid sequence with SEQ ID NO: 3. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 4. In some embodiments, the IL-15 domain comprises an amino acid sequence with SEQ ID NO: 4. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 10. In some embodiments, the IL-15 domain comprises an amino acid sequence with SEQ ID NO: 10.

In some embodiments, the bispecific molecule is conjugated to a histone deacetylases (HDAC) inhibitor. In some embodiments, the HDAC inhibitor is Romidepsin (RMD). In some embodiments, the bispecific molecule is conjugated to a PKC activator. In some embodiments, the PKC activator is Ingenol mebutate. In some embodiments, the bispecific molecule is conjugated to a PTEN inhibitor. In some embodiments, the PTEN inhibitor is Disulfiram. In some embodiments, the bispecific molecule is conjugated to a RIG-1 activator. In some embodiments, the RIG-1 activator is Acitretin. In some embodiments, the bispecific molecule is conjugated to a SMAC mimetic. In some embodiments, the SMAC mimetic is Birinapant. In some embodiments, the bispecific molecule is T4IL15. In some embodiments, the bispecific molecule is T4IL15(-Rα). In some embodiments, the method further comprises administering to the subject antiretroviral therapy. In some embodiments, the antiretroviral comprises 10E8.4/iMab administration. In some embodiments, the antiretroviral comprises Islatravir administration.

In certain aspects, the subject matter described herein provides a method of reactivating a HIV-1 reservoir in a subject in need thereof, the method comprising administering to the subject a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide and a fourth polypeptide, wherein the third polypeptide comprises CH2-CH3 domains of a heavy chain and the fourth polypeptide comprises an IL-15 domain, wherein the CH2-CH3 domains of the heavy chain are coupled to IL-15 domain; and wherein the heavy chain of the first arm and the Ch2-CH3 domains of the second arm form a knob-into-hole structure.

In some embodiments, the first arm forms a first antigen binding region. In some embodiments, the first antigen binding region binds CD4. In some embodiments, the second arm forms a second antigen binding region. In some embodiments, the second antigen binding region binds an IL-15 receptor. In some embodiments, the heavy chain comprises CH1-CH3 constant domains and a variable domain, wherein the CH2 domain is linked to the CH1 via a GSx3 linker. In some embodiments, the CH2-CH3 domains are the IgG1 Fc CH2-CH3 domains. In some embodiments, the IgG Fc domain comprises LALA mutations. In some embodiments, the heavy chain of the first arm is coupled to the light chain of the first arm via one or more disulfide bonds. In some embodiments, the CH2-CH3 domains of the second arm are coupled to an IL-15 domain via a GSx3 linker. In some embodiments, the heavy chain of the first arm is coupled the CH2-CH3 domain of the second arm via one or more disulfide bonds. In some embodiments, the bispecific molecule further comprises a Sushi domain of an IL-15 receptor α chain. In some embodiments, the Sushi domain is coupled to the CH2-CH3 domains of the second arm via a GSx3 linker. In some embodiments, the Sushi domain is coupled to the IL-15 domain via one or more disulfide bonds. In some embodiments, the CH2-CH3 domains of the second arm are not directly coupled to the IL-15 domain.

In some embodiments, the light chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the light chain of the first arm comprises an amino acid sequence with SEQ ID NO: 1. In some embodiments, the light chain of the first arm comprises a variable (VL) domain and a constant (CL) domain, wherein the VL comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 9. In some embodiments, the light chain of the first arm comprises a variable (VL) domain and a constant (CL) domain, wherein the VL comprises an amino acid sequence with SEQ ID NO: 9. In some embodiments, the heavy chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 2. In some embodiments, the heavy chain of the first arm comprises an amino acid sequence with SEQ ID NO: 2. In some embodiments, the second arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 3. In some embodiments, the second arm comprises an amino acid sequence with SEQ ID NO: 3. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 4. In some embodiments, the IL-15 domain comprises an amino acid sequence with SEQ ID NO: 4. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 10. In some embodiments, the IL-15 domain comprises an amino acid sequence with SEQ ID NO: 10.

In some embodiments, the bispecific molecule is conjugated to a histone deacetylases (HDAC) inhibitor. In some embodiments, the HDAC inhibitor is Romidepsin (RMD). In some embodiments, the bispecific molecule is conjugated to a PKC activator. In some embodiments, the PKC activator is Ingenol mebutate. In some embodiments, the bispecific molecule is conjugated to a PTEN inhibitor. In some embodiments, the PTEN inhibitor is Disulfiram. In some embodiments, the bispecific molecule is conjugated to a RIG-1 activator. In some embodiments, the RIG-1 activator is Acitretin. In some embodiments, the bispecific molecule is conjugated to a SMAC mimetic. In some embodiments, the SMAC mimetic is Birinapant. In some embodiments, the bispecific molecule is T4IL15. In some embodiments, the bispecific molecule is T4IL15(-Rα). In some embodiments, the method further comprises administering to the subject antiretroviral therapy. In some embodiments, the antiretroviral comprises 10E8.4/iMab administration. In some embodiments, the antiretroviral comprises Islatravir administration. In some embodiments, the reservoir is a latent reservoir. In some embodiments, the reservoir comprises HIV-1 infected CD4+ T cells.

In certain aspects, the subject matter described herein provides a bispecific molecule means for binding IL15 Receptor and CD4. In some embodiments, the means comprises any one of the bispecific molecules of claim 1-41. In some embodiments, the means is capable of treating a HIV-1 infection in a subject in need thereof or reactivating a HIV-1 reservoir in a subject in need thereof.

### BRIEF DESCRIPTION OF THE FIGURES

The patent or application file contains at least one drawing executed in color. Copies of this patent or patent application publication with color drawing(s) will be provided by the Office upon request and payment of the necessary fec.

FIGS. 1A-C show a bispecific molecule disclosed here (T4IL15). The molecule has a first arm with a first antigen binding region which binds a CD4. The molecule also has a second arm with a second antigen binding region which binds an IL-15 receptor. FIG. 1A shows a T4IL15 design. FIG. 1B shows a Size Exclusion Chromatography (SEC) profile for T4IL15. FIG. 1C shows a bispecific molecule disclosed here (T4IL15) with a Romidepsin (RMD) molecule conjugated to T4IL15 (T4IL15-RMD) design. Black lines indicated by “#” are GSx3 linker between IL5Ra and Fc of IgG. Red line indicated by “*” are disulfide bonds. KIH=knob-into-hole format. As shown in FIGS. 1A and 1C, the heavy chain of the first arm comprises a variable domain (VH) and three constant domains (CH1-CH3). The light chain of the first arm comprises one variable domain (VL) and one constant domain (CL).

FIGS. 2A-F show ex vivo evaluation of a bispecific molecule (T4IL15) activity in healthy donor PBMCs. FIG. 2A shows that 5×10⁵ PBMCs from each donor (150 μL, n=12) were stimulated with the respective test articles (TAs). TA concentration: 0.1 and 0.02 μg/mL. Stimulation for ˜18 hours. FIG. 2B shows an N-803 design and an IL15dsFc design. FIG. 2C shows CD69 upregulation of CD4+ T cells measured ˜18 hours post stimulation. FIGS. 2D-E show that IL15dsFc/iMab activates CD45RO+ memory CD8+ T cells, but not CD45RO-naïve CD8+ T cells with 0.1 μg/ml (FIG. 2D) or 0.002 μg/ml (FIG. 3E). 2F shows that T4IL15 retains the ability to activate NK cells.

FIGS. 3A-D show a time course of CD69 expression on CD4+ T cells following stimulation with T4IL15. FIG. 3A shows an increase in CD69+ cells with T4IL15 stimulation over 7 days. The CD45RO antigen, an isoform of CD45 antigen, is a marker of memory T cells, which proliferate in response to recall antigen. By the expression of the CD45RO antigen, CD4+ T cells are sub-grouped into CD45RO-positive memory CD4+ T cells and CD45RO-negative naive CD4+ T cells. FIG. 3B indicates the histogram for the CD69 signal and shows the fraction of CD69 positive cells for CD45RO+CD4+ T cells and CD45RO-CD4+ T cells at day 7 following stimulation with T4IL15. Similarly, FIG. 3C shows the fraction of CD69 positive cells for CD45RO+CD4+ T cells and CD45RO-CD4+ T cells at day 7 without stimulation (negative control). FIG. 3D shows the fraction of CD69 positive cells for CD45RO+CD4+ T cells and CD45RO-CD4+ T cells at 7 days following stimulation by the Dynabeads-CD3/CD28 Antibody (positive control).

FIG. 4 shows that T4IL15 activates HIV-1 latently infected cells in vitro. Purified CD4+ cells (2×10⁶ cells/well) from 8 HIV-infected well-suppressed patients were stimulated with T4IL15 (0.1 μg/mL). Intracellular HIV RNA expressions (RNA copics/well) were quantified at day 3 and day 8. Unstimulated negative intracellular HIV-RNA value (0 copy) was assigned as 1 copy for fold induction calculation. Over 5-fold induction scored as measurable latent reservoir reactivation. Culture supernatant was harvested for replication competent virus detection using TZA assay. All samples shown were stimulated with T4IL15. The unstimulated controls were used as background (0 copy).

FIG. 5 shows viral suppression using Islatravir and 10E8.4/iMabN297A (QW) in 8 infected hu NSG mice. A weekly regimen of 40 mg/kg of Islatravir in combination with 20 mg/kg of 10E8.4/iMab initial dose followed by 10 mg/kg of weekly doses in huNSG infected with HIV-AD8 virus. Blood plasma was collected prior to each injection and viral load was measured from extracted RNA using qRT-PCR. The huNSG mice achieved full suppression of virus by week 7 and stayed aviremic for 6 continuous weeks of treatment (FIG. 5). At the end of the study, all humanized mice with full virus suppression maintained normal peripheral CD4 T cell counts and CD4+ T cell subset phenotype profiles were similar to human PBMCs (data not shown). The sustained viral suppression seen in this case is likely due to potential synergy effect of the combination of bispecific antibody with Islatravir.

FIGS. 6A-B show selectivity of T4IL15. FIG. 6A shows IL15dsFc and T4IL15 activation data from FIGS. 2C and 2D. FIG. 6B shows that IL15dsFc has a selectivity index of 0.56, whereas T4IL15 has an index of 0.79.

FIGS. 7A-C show IL15 design modifications to enhance selectivity. FIG. 7A shows T4IL15 bispecific construct without an IL15 and Sushi domain receptor α complex (called T4IL15(-Rα)). T4IL15(-Rα) comprises IL15-GSx3 CH2-CH3 knob (SEQ ID NO: 11), MV1 HC hole (SEQ ID NO: 12), and MV1 LC (SEQ ID NO: 13). As shown in FIGS. 1A and 1C, the heavy chain of the first arm comprises a variable domain (VH) and three constant domains (CH1-CH3). The light chain of the first arm comprises one variable domain (VL) and one constant domain (CL). FIG. 7B shows T4IL15(-Rα) activation of CD4+ T cells. FIG. 7C shows reduced memory CD8+ T cell activation by T4IL15(-Rα).

FIGS. 8A-E show IL15 mutant with reduced binding affinity and improved selectivity. FIG. 8A shows in silico modeling of interactions between IL15 cytokine with IL2 receptor β and common chain γ. FIG. 8B shows identification of these interactions. FIG. 8C shows constructs evaluated by HEK-Blue Report assay. FIG. 8D shows activation of memory CD4 or memory CD8 cells in human PBMC-based CD69 upregulation following candidate administration. FIG. 8E shows improved selectivity index for T4IL15(D8A).

FIGS. 9A-C show modifications to the CD4 binding arm of the bispecific molecules described here to further enhance selectivity. FIG. 9A shows reduced binding affinity to CD4 by T4Δ(LC.Y32H)IL15 construct as compared to parental T4IL15 in surface plasmon resonance (SPR) binding analysis. FIG. 9B shows T4ΔIL15(D8A) retained memory CD4+ T cell activation at comparable levels to parental T4IL15. FIG. 9C shows T4ΔIL15(D8A) reduced memory CD8+ T cell activation, thereby increasing the selectivity index to 7.21.

FIG. 10 shows reactivation of HIV-1 provirus in patient PBMCs receiving engineered bispecific molecules. PBMC from patients with controlled viremia on suppressive antiretroviral therapy (ART) were treated directly with each of the embodiments and comparators (IL15RadsFc, T4IL15 and T4ΔIL15D8A) and an irrelevant CD4-targeting bispecific antibody at 0.1 ug/mL dose for a period of 5 days before the supernatant was collected and centrifuged for viral particle isolation. Phorbol myristoyl acetate (50 ng/ml) combined with 1 uM ionomycin (PMA/Io) was used as a positive control for the virus reactivation assay based on previously published studies. After 5 days of incubation, viral particles were pelleted from the supernatant for RNA extraction and detected using RT-qPCR.

FIGS. 11A-11B show reactivation of HIV-1 provirus in ART suppressed aviremic humanized mice using engineered bispecific molecules. FIG. 11A shows an experimental approach for testing the ability of an intervention to reactivate latent HIV-1 provirus in humanized mouse models. NSG mice were reconstituted with human hematopoietic stem cells and subsequently infected with HIV-1 strain AD8 at a dose of 30,000 TCID50. Following steady-state infection, the mice were treated with a cocktail of bispecific antibodies (10E8.4/iMab 297A and PGT121/VRC07-LS) on a weekly basis to achieve complete suppression of active viral replication. Once complete suppression was achieved, the mice were kept on maintenance therapy throughout the remaining period of experimentation. FIG. 11B shows the cumulative frequency and magnitude of reactivation of HIV-1 in ART suppressed aviremic humanized mice. The study was initiated by injecting mice with indicated doses: (3/6 μg) of IL15RadsFc or 3 μg of T4IL15 or 3 μg of T4ΔIL15(D8A). A total of 9 doses were tested for IL15RadsFc (3 μg) and 16 doses of each IL15RadsFc (6 μg), T4IL15 (3 μg) and T4ΔIL15(D8A) (3 μg). Two days post each dosing; mice were bled for extracting plasma RNA to measure levels of HIV-1 using one-step RT-PCR that amplified the levels of the Pol gene in the blood and served as a marker to indicate viral reactivation. Viral blips are quantitated as copies/mL based on a standard curve run in parallel to the samples.

### DETAILED DESCRIPTION OF THE INVENTION

In certain aspects, the subject matter described herein provides a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide and a fourth polypeptide, wherein the third polypeptide comprises CH2-CH3 domains of a heavy chain and the fourth polypeptide comprises an IL-15 domain, wherein the CH2-CH3 domains of the heavy chain are coupled to IL-15 domain; and wherein the heavy chain of the first arm and the Ch2-CH3 domains of the second arm form a knob-into-hole structure.

In some embodiments, the first arm forms a first antigen binding region. In some embodiments, the first arm binds CD4. In some embodiments, the first arm binds CD4 with a first antigen binding region. In some embodiments, the second arm forms a second antigen binding region. In some embodiments, the second arm binds an IL-15 receptor. In some embodiments, the second arm binds an IL-15 receptor with the second binding region. In some embodiments, the second arm binds an IL2/IL-15 receptor β/γ heterodimer. Without being bound by theory, IL-15 ligand binds IL-15 receptor-α to form a IL15/IL15Rα complex, which then binds the IL2/IL-15 receptor β/γ heterodimer. The IL2/IL-15 receptor β/γ heterodimer is also capable of binding IL-2 ligand. In some embodiments, the heavy chain comprises CH1-CH3 constant domains and a variable domain, wherein the CH2 domain is linked to the CH1 via a GSx3 linker. In some embodiments, the CH2-CH3 domains are the IgG1 Fc CH2-CH3 domains. In some embodiments, the IgG Fc domain comprises LALA mutations. In some embodiments, the heavy chain of the first arm is coupled to the light chain of the first arm via one or more disulfide bonds. In some embodiments, the CH2-CH3 domains of the second arm are coupled to an IL-15 domain via a GSx3 linker. In some embodiments, the heavy chain of the first arm is coupled the CH2-CH3 domain of the second arm via one or more disulfide bonds. In some embodiments, the bispecific molecule further comprises a Sushi domain of an IL-15 receptor α chain. A sushi domain is a conserved protein domain comprising a beta-sandwich arrangement wherein one face of the domain comprises three β-strands hydrogen-bonded to form a triple-stranded region at its center, and the other face formed from two separate β-strands. The sushi domain of an IL-15 receptor α chain is described, for example, in Wei et al., Journal of Immunol., Volume 167, Issue 1, July 2001, pages 277-282, the content of which is herein incorporated by reference in its entirety. In some embodiments, the Sushi domain is coupled to the CH2-CH3 domains of the second arm via a GSx3 linker. In some embodiments, the Sushi domain is coupled to the IL-15 domain via one or more disulfide bonds. In some embodiments, the CH2-CH3 domains of the second arm are not directly coupled to the IL-15 domain.

In some embodiments, the light chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the light chain of the first arm comprises an amino acid sequence with SEQ ID NO: 1. In some embodiments, the light chain of the first arm comprises a variable (VL) domain and a constant (CL) domain, wherein the VL comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 9. In some embodiments, the light chain of the first arm comprises a variable (VL) domain and a constant (CL) domain, wherein the VL comprises an amino acid sequence with SEQ ID NO: 9. In some embodiments, the heavy chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 2. In some embodiments, the heavy chain of the first arm comprises an amino acid sequence with SEQ ID NO: 2. In some embodiments, the second arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 3. In some embodiments, the second arm comprises an amino acid sequence with SEQ ID NO: 3. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 4. In some embodiments, the IL-15 domain comprises an amino acid sequence with SEQ ID NO: 4. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 10. In some embodiments, the IL-15 domain comprises an amino acid sequence with SEQ ID NO: 10.

In some embodiments, the bispecific molecule is conjugated to a histone deacetylases (HDAC) inhibitor. In some embodiments, the HDAC inhibitor is Romidepsin (RMD). In some embodiments, the bispecific molecule is conjugated to a PKC activator. In some embodiments, the PKC activator is Ingenol mebutate. In some embodiments, the bispecific molecule is conjugated to a PTEN inhibitor. In some embodiments, the PTEN inhibitor is Disulfiram. In some embodiments, the bispecific molecule is conjugated to a RIG-1 activator. In some embodiments, the RIG-1 activator is Acitretin. In some embodiments, the bispecific molecule is conjugated to a SMAC mimetic. In some embodiments, the SMAC mimetic is Birinapant. In some embodiments, the bispecific molecule is T4IL15. In some embodiments, the bispecific molecule is T4IL15(-Rα).

In certain aspects, the subject matter described herein provides a pharmaceutical composition comprising any of the bispecific molecule disclosed herein.

In certain aspects, the subject matter described herein provides a polynucleotide encoding any of the bispecific molecule disclosed herein or a fragment thereof.

In certain aspects, the subject matter described herein provides a virus comprising a polynucleotide encoding any of the bispecific molecule disclosed herein or a fragment thereof.

In certain aspects, the subject matter described herein provides a genetically engineered cell comprising any of the bispecific molecule disclosed herein.

In certain aspects, the subject matter described herein provides a genetically engineered cell comprising any of the bispecific molecule disclosed herein or a fragment thereof.

In certain aspects, the subject matter described herein provides a method of treating latent HIV-1 infection in a subject in need thereof, the method comprising administering to the subject a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide and a fourth polypeptide, wherein the third polypeptide comprises CH2-CH3 domains of a heavy chain and the fourth polypeptide comprises an IL-15 domain, wherein the CH2-CH3 domains of the heavy chain are coupled to IL-15 domain; and wherein the heavy chain of the first arm and the Ch2-CH3 domains of the second arm form a knob-into-hole structure.

In some embodiments, the first arm forms a first antigen binding region. In some embodiments, the first antigen binding region binds CD4. In some embodiments, the second arm forms a second antigen binding region. In some embodiments, the second antigen binding region binds an IL-15 receptor. In some embodiments, the heavy chain comprises CH1-CH3 constant domains and a variable domain, wherein the CH2 domain is linked to the CH1 via a GSx3 linker. In some embodiments, the CH2-CH3 domains are the IgG1 Fc CH2-CH3 domains. In some embodiments, the IgG Fc domain comprises LALA mutations. In some embodiments, the heavy chain of the first arm is coupled to the light chain of the first arm via one or more disulfide bonds. In some embodiments, the CH2-CH3 domains of the second arm are coupled to an IL-15 domain via a GSx3 linker. In some embodiments, the heavy chain of the first arm is coupled to the CH2-CH3 domain of the second arm via one or more disulfide bonds. In some embodiments, the bispecific molecule further comprises a Sushi domain of an IL-15 receptor α chain. In some embodiments, the Sushi domain is coupled to the CH2-CH3 domains of the second arm via a GSx3 linker. In some embodiments, the Sushi domain is coupled to the IL-15 domain via one or more disulfide bonds. In some embodiments, the CH2-CH3 domains of the second arm are not directly coupled to the IL-15 domain.

In some embodiments, the light chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the light chain of the first arm comprises an amino acid sequence with SEQ ID NO: 1. In some embodiments, the light chain of the first arm comprises a variable (VL) domain and a constant (CL) domain, wherein the VL comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 9. In some embodiments, the light chain of the first arm comprises a variable (VL) domain and a constant (CL) domain, wherein the VL comprises an amino acid sequence with SEQ ID NO: 9. In some embodiments, the heavy chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 2. In some embodiments, the heavy chain of the first arm comprises an amino acid sequence with SEQ ID NO: 2. In some embodiments, the second arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 3. In some embodiments, the second arm comprises an amino acid sequence with SEQ ID NO: 3. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 4. In some embodiments, the IL-15 domain comprises an amino acid sequence with SEQ ID NO: 4. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 10. In some embodiments, the IL-15 domain comprises an amino acid sequence with SEQ ID NO: 10.

In some embodiments, the bispecific molecule is conjugated to a histone deacetylases (HDAC) inhibitor. In some embodiments, the HDAC inhibitor is Romidepsin (RMD). In some embodiments, the bispecific molecule is conjugated to a PKC activator. In some embodiments, the PKC activator is Ingenol mebutate. In some embodiments, the bispecific molecule is conjugated to a PTEN inhibitor. In some embodiments, the PTEN inhibitor is Disulfiram. In some embodiments, the bispecific molecule is conjugated to a RIG-1 activator. In some embodiments, the RIG-1 activator is Acitretin. In some embodiments, the bispecific molecule is conjugated to a SMAC mimetic. In some embodiments, the SMAC mimetic is Birinapant. In some embodiments, the bispecific molecule is T4IL15. In some embodiments, the bispecific molecule is T4IL15(-Rα). In some embodiments, the method further comprises administering to the subject antiretroviral therapy. In some embodiments, the antiretroviral comprises 10E8.4/iMab administration. In some embodiments, the antiretroviral comprises Islatravir administration.

In certain aspects, the subject matter described herein provides a method of reactivating a HIV-1 reservoir in a subject in need thereof, the method comprising administering to the subject a bispecific molecule comprising: a first arm comprising a first polypeptide and a second polypeptide, wherein the first polypeptide comprises a heavy chain and the second polypeptide comprises a light chain, wherein the heavy chain is coupled to the light chain, a second arm comprising a third polypeptide and a fourth polypeptide, wherein the third polypeptide comprises CH2-CH3 domains of a heavy chain and the fourth polypeptide comprises an IL-15 domain, wherein the CH2-CH3 domains of the heavy chain are coupled to IL-15 domain; and wherein the heavy chain of the first arm and the Ch2-CH3 domains of the second arm form a knob-into-hole structure.

In some embodiments, the first arm forms a first antigen binding region. In some embodiments, the first antigen binding region binds CD4. In some embodiments, the second arm forms a second antigen binding region. In some embodiments, the second antigen binding region binds an IL-15 receptor. In some embodiments, the heavy chain comprises CH1-CH3 constant domains and a variable domain, wherein the CH2 domain is linked to the CH1 via a GSx3 linker. In some embodiments, the CH2-CH3 domains are the IgG1 Fc CH2-CH3 domains. In some embodiments, the IgG Fc domain comprises LALA mutations. In some embodiments, the heavy chain of the first arm is coupled to the light chain of the first arm via one or more disulfide bonds. In some embodiments, the CH2-CH3 domains of the second arm are coupled to an IL-15 domain via a GSx3 linker. In some embodiments, the heavy chain of the first arm is coupled the CH2-CH3 domain of the second arm via one or more disulfide bonds. In some embodiments, the bispecific molecule further comprises a Sushi domain of an IL-15 receptor α chain. In some embodiments, the Sushi domain is coupled to the CH2-CH3 domains of the second arm via a GSx3 linker. In some embodiments, the Sushi domain is coupled to the IL-15 domain via one or more disulfide bonds. In some embodiments, the CH2-CH3 domains of the second arm are not directly coupled to the IL-15 domain.

In some embodiments, the light chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 1. In some embodiments, the light chain of the first arm comprises an amino acid sequence with SEQ ID NO: 1. In some embodiments, the light chain of the first arm comprises a variable (VL) domain and a constant (CL) domain, wherein the VL comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 9. In some embodiments, the light chain of the first arm comprises a variable (VL) domain and a constant (CL) domain, wherein the VL comprises an amino acid sequence with SEQ ID NO: 9. In some embodiments, the heavy chain of the first arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 2. In some embodiments, the heavy chain of the first arm comprises an amino acid sequence with SEQ ID NO: 2. In some embodiments, the second arm comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 3. In some embodiments, the second arm comprises an amino acid sequence with SEQ ID NO: 3. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 4. In some embodiments, the IL-15 domain comprises an amino acid sequence with SEQ ID NO: 4. In some embodiments, the IL-15 domain comprises an amino acid sequence with at least 80%, 81%, 82%, 83%, 84%, 85%, 86%, 87%, 88%, 89%, 90%, 91%, 92%, 93%, 94%, 95%, 96%, 97%, 98%, 99% identity to SEQ ID NO: 10. In some embodiments, the IL-15 domain comprises an amino acid sequence with SEQ ID NO: 10.

In some embodiments, the bispecific molecule is conjugated to a histone deacetylases (HDAC) inhibitor. In some embodiments, the HDAC inhibitor is Romidepsin (RMD). In some embodiments, the bispecific molecule is conjugated to a PKC activator. In some embodiments, the PKC activator is Ingenol mebutate. In some embodiments, the bispecific molecule is conjugated to a PTEN inhibitor. In some embodiments, the PTEN inhibitor is Disulfiram. In some embodiments, the bispecific molecule is conjugated to a RIG-1 activator. In some embodiments, the RIG-1 activator is Acitretin. In some embodiments, the bispecific molecule is conjugated to a SMAC mimetic. In some embodiments, the SMAC mimetic is Birinapant. In some embodiments, the bispecific molecule is T4IL15. In some embodiments, the bispecific molecule is T4IL15(-Rα). In some embodiments, the method further comprises administering to the subject antiretroviral therapy. In some embodiments, the antiretroviral comprises 10E8.4/iMab administration. In some embodiments, the antiretroviral comprises Islatravir administration. In some embodiments, the reservoir is a latent reservoir. In some embodiments, the reservoir comprises HIV-1 infected CD4+ T cells.

In certain aspects, the subject matter described herein provides a bispecific molecule means for binding IL15 Receptor and CD4. In some embodiments, the means comprises any one of the bispecific molecules of claim 1-41. In some embodiments, the means is capable of treating a HIV-1 infection in a subject in need thereof or reactivating a HIV-1 reservoir in a subject in need thereof.

### Antibodies

In some embodiments, the bispecific molecule comprises an antibody or functional fragment thereof. There are five classes of human antibodies (i.e., IgA, IgD, IgE, IgG, and IgM) and each have various isotypes (e.g., IgG1, IgG2, IgG3, IgG4, IgAQ1, and IgA2). In some embodiments, the antibodies disclosed herein belong to the IgG class. IgG can be further divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety.

The IgG immunoglobulin molecule consists of four polypeptide chains, two identical light (L) chains and two identical heavy (H) chains. The four chains are joined by disulfide bonds in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy-chain variable domains. The more highly conserved portions of variable domains are called the framework regions (FR). The variable domains of native heavy and light chains each comprise four FRs, largely adopting a beta-sheet configuration, connected by three CDRs, which form loops connecting, and in some cases forming part of, the beta-sheet structure. The CDRs in each chain are held together in close proximity by the FRs and, with the CDRs from the other chain, contribute to the formation of the antigen binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibody to an antigen, but exhibit various effector functions, such as participation of the antibody in antibody-dependent cellular toxicity.

### HIV-1

HIV (human immunodeficiency virus) attacks the body's immune system and, if the HIV infection is left untreated, it can lead to AIDS (acquired immunodeficiency syndrome).

There is currently no effective cure for an HIV infection, however, with proper medical care, HIV can be controlled. There are typically three stages of HIV infection. Stage 1 is the acute phase of the infection. During this stage, patients have a large amount of the HIV virus in their blood and they are very contagious. Often patients develop flu-like symptoms. Stage 2 is when patients have a chronic HIV infection. During this stage the patient can be asymptomatic and the HIV infection can be into clinical latency. Patients may not have any symptoms or get sick during this stage but they can transmit HIV and the virus is still active and reproduces in the body. The end of this stage is when the amount of HIV in the blood (viral load) increases dramatically. Patients on HIV treatment may never transition from Stage 2 into Stage 3, which is characterized by the development of AIDS. This is the most severe stage of an HIV infection with a high viral load and may easily transmit HIV to others. The survival expectancy at this stage is about three years.

HIV attacks immune system cells in the subject's body and uses the replication machinery available in the subject's cells to replicate itself. HIV-infected immune cells can go into a resting or latent state during which the infected cells do not produce new viral particles. Using the latent mechanism, the virus can reside undetected inside the subject's immune cells for years, forming a latent HIV reservoir. At any time, the infected cells in the latent reservoir can become active again and start making more viral particles, accelerating the disease.

The HIV-1 virus can establish a latent reservoir during primary infection. The reservoir comprises primarily of HIV-infected and long-lived subpopulations of CD4+ resting memory T cells. The available HIV medicines prevent the virus from multiplying, which reduces the amount of the virus in the body (i.e., the viral load). However, HIV medicines have no effect on HIV-infected cells in a latent reservoir because they are not producing new copies of the virus. HIV positive patients must take a daily combination of anti-HIV medications to keep their viral loads low. If they stop the anti-HIV medications, the infected cells from the latent reservoir can begin making HIV again and the patient's viral load will increase. While currently available antiretroviral therapies (ART) can reduce the level of HIV-1 in blood to an undetectable level, ARTs also cannot eliminate the latent reservoir, thereby imposing a major obstacle to curing the infection. Therefore, developing strategies to eliminate or to reduce the viral reservoir that could lead to a cure or lifelong remission of HIV-1 infection remains a key priority in HIV/AIDS research.

### Immune Cells Affected by HIV Infections

HIV virus weakens the infected subject's immune system by destroying T4 lymphocytes, also called CD4+ T lymphocytes (CD4+ T cells). CD4+ T cells coordinate the immune response by stimulating/activating other immune cells, such as macrophages, B lymphocytes (B cells), and CD8+ T lymphocytes (CD8 T cells). This repertoire of immune cells fights viral infections such as an HIV-1 infection. HIV infects the T cells via a high-affinity interaction between the virion envelope glycoprotein (gp120) and the CD4 molecule on the surface of the T cells. This interaction is assisted by a T-cell co-receptor, CXCR4. After gp120 binds to CD4 on the T cell, nucleocapsids containing viral genome and enzymes enters the target cell, which becomes the host cell for viral reproduction. Following the release of viral genome and enzymes from the core protein, viral reverse transcriptase catalyzes reverse transcription of viral single strand RNA to form RNA-DNA hybrid complexed. To generate the HIV encoding double stranded DNA, the viral RNA template is partially degraded by ribonuclease H and the second DNA (dsDNA) strand is synthesized. The viral dsDNA is translocated into the host cell nucleus and it is integrated into the host genome by the viral integrase enzyme. Transcription factors transcribe the proviral DNA into genomic ssRNA. The ssRNA is then exported to the cytoplasm where host-cell ribosomes catalyze synthesis of viral precursor proteins which are cleaved into viral proteins by viral proteases. HIV ssRNA and proteins assemble within the host-cell forming virions. The mature virions are able to infect another host cell.

During an HIV infection, CD8+ T-cells recognize infected cells through an MHC-I dependent process and lyse cells harboring viral infection. The lysing can be achieved by the secretion of perforin and granzymes. CD8+ T-cells can also eliminate virally infected cells through the engagement of death-inducing ligands expressed by CD8+ T-cells with death receptors on the surface of the infected cell. Additionally, CD8+ cells secrete soluble factors such as beta-chemokines and the CD8+ antiviral factor (CAF) that suppress viral binding and transcription.

For the HIV virus to evade the survival mechanism of the host immune system, the virus has adopted numerous strategies to evade the CD8+ T-cell response. For example, HIV has a high mutation rate, which allows the virus to escape CD8+ T-cell recognition in addition down-regulating surface MHC-I expression from infected cells. Also, HIV can disrupt proper CD8+ T-cell signaling by altering the pattern of cytokine production. Overall, HIV is able to decrease the circulating pool of effector and memory CD8+ T-cells that are able to combat viral infection by affecting the function of CD4+ T-cells and antigen presenting cells that are required for proper CD8+ T-cell maturation.

### Interleukin 15

Interleukin 15 (IL-15 or IL15) is an inflammatory cytokine of about 12-14 KD produced by antigen-presenting cells. IL-15 induces selective activation and proliferation of natural killer (NK) cells and T cells. IL-15 can promote both innate and adaptive immune reactions by stimulating CD8+/CD4+ T cells and natural killer cells (NK). An IL-15-based molecule has been generated to take advantage of the drug-like properties of IL-15. More details on the IL-15-based molecule can be found in Hu, Q. et al., “Discovery of a novel IL-15 based protein with improved developability and efficacy for cancer immunotherapy” Scientific Reports volume 8, Article number: 7675 (2018), the contents of which is incorporated herein by reference in its entirety. The molecule comprises a complex of IL-15 and the Sushi domain of the IL-15 receptor α chain, which enhances the agonist activity of IL-15 via trans-presentation, a disulfide bond linking the IL-15/Sushi domain complex with an IgG1 Fc to increase its half-life. IL-15 binds to the IL-15 receptor (IL-15R) which consists of α, β, and γ_{c} chains. IL-15Rα is widely expressed and binds IL-15 with high affinity. IL-15Rα contains a Sushi domain (1-65 amino acids), which is responsible for the receptor's interaction with the IL-15 ligand, and is essential for mediating the biological function of IL-15. Natural IL-15 has druggability problems, despite its potential for therapeutic use. These include low biological potency and a short half-life. To develop an IL-15-based therapeutic agent, it is important to enhance the IL-15 potency and extend its half-life. The complex formed by IL-15 and the Sushi domain of soluble IL-15Rα is significantly stronger than IL-15 alone in stimulating the proliferation of T lymphocytes and NK cells. A covalent linkage formed between the IL-15 and the Sushi domain by one or more disulfide bonds can significantly increase the stability of the complex due to the decrease in the entropy of the unfolded protein. Mutations such as L52C of IL-15 and S40C of IL-15Rα facilitate pairing of the disulfide bonds and protein stability. To improve the half-life of the IL-15 and the Sushi domain complex, the Sushi domain can be associated or fused with the Fragment Crystallizable region (Fc) of a human IgG1.

IL-15 has been shown to activate HIV-1 in infected memory CD4+ T cells expressing the IL-15 receptor by producing viral gene transcripts in vitro. Due to its potential in activating the latent reservoir while enhancing the effector function of host immune responses, one recent phase I clinical trial to assess the safety and virologic impact of the IL-15 super-agonist N-803 (IL15RaFc) in people living with ART suppressed HIV was reported (Miller, J. S. et al. 2022. Safety and virologic impact of the IL-15 superagonist N-803 in people living with HIV: a phase 1 trial. Nat Med. 2022 February; 28 (2): 392-400). This study showed that 1) N-803 was safe and well-tolerated at the tested doses, 2) the administration of N-803 was associated with proliferation and activation of CD4+, CD8+ T cells, and NK cells, and 3) a modest reduction in the inducible HIV-1 reservoir was observed in PBMCs from participants receiving the agonist.

In some embodiments the subject matter described herein relates to methods of treating an HIV infection in a subject in need thereof, by administering to the subject a bispecific molecule comprising a second arm comprising IL-15. In some embodiments, the subject matter described herein relates to methods of treating an HIV infection in a subject in need thereof, by administering to the subject a bispecific molecule comprising a second arm comprising an IL-15 and Sushi domain of receptor α chain complex. In some embodiments, the IL-15 or the IL-15 and Sushi domain of receptor α chain complex are each associated with or fused to an IgG1 Fc. In some embodiments, the bispecific molecule comprises a first arm comprising one or more iMab domains.

In some embodiments the subject matter described herein relates to methods of reactivating a latent HIV reservoir in a subject in need thereof, by administering to the subject a bispecific molecule comprising a second arm comprising IL-15. In some embodiments, the subject matter described herein relates to methods of reactivating a latent HIV reservoir in a subject in need thereof, by administering to the subject a bispecific molecule comprising a second arm comprising an IL-15 and Sushi domain of receptor α chain complex. In some embodiments, the IL-15 or the IL-15 and Sushi domain of receptor α chain complex are each associated with or fused to an IgG1 Fc. In some embodiments, the bispecific molecule comprises a first arm comprising one or more iMab domains.

### Latency Reversal Agents (LRAs)

Among the strategies being pursued toward eliminating the latent reservoirs, the “Shock-and-Kill” approach, aims to induce HIV-1 expression from latent infected cells using latency reversal agents (LRAs). LRAs reactivate latent HIV within CD4+ cells, allowing ART and the body's immune system to attack the virus. HIV-1 reactivation can facilitate the clearance of these cells either by a viral cytopathic effect, or by host immune responses, with the ultimate goal of reducing the size of the viral reservoirs or completely eliminating them. Viral cytopathic effects include morphological changes in host cells caused by viral infection.

A number of small molecule LRAs, such as histone deacetylase (HDAC) inhibitors and Protein Kinase C (PKC) activators, have shown viral reactivation in vitro in generating viral transcripts or virus-like particles, but they have little or no impact on the reduction of latent reservoirs in physiologically relevant concentration in clinical trials. Increasing the dose of LRAs is prohibitive in their current forms due to the potential systemic toxicity.

In some embodiments, the subject matter described herein relates to targeted delivering of LRAs. In some embodiments, the subject matter described herein relates to the specificity of monoclonal antibodies to CD4+ T cells. Without being bound to theory, a bispecific molecule targeting both CD4 and IL-15 receptor simultaneously will selectively target CD4+ T cells. In some embodiments, the subject matter described herein relates to more effective and specific activation of the resting memory CD4+ T cell population. In some embodiments, the resting memory CD4+ T cell population is a major HIV-1 reservoir in blood and tissues of infected subjects.

Ibalizumab (iMab)

Ibalizumab (iMab) is a humanized IgG4 monoclonal antibody that binds human CD4, which is the primary receptor for HIV-1. iMab blocks entry of HIV-1 into the target cells. The antibody is derived from mouse MAB 5A8 and binds CD4 extracellular domain 2. It prevents conformational changes in the CD4-HIV envelope glycoprotein (gp120) complex that are important for viral entry.

In some embodiments, the subject matter described herein relates to a bispecific molecule with one arm containing an anti-CD4 monoclonal antibody (mAb) iMab (Ibalizumab) and the other arm containing an IL-15 or IL-15 and Sushi domain of receptor α chain complex which is capable of targeting the IL-2/IL-15 receptor IL-2Rβ (CD122) and the common gamma chain (γC, CD132). CD122, CD132, and IL-12Rα comprise the IL-15 receptor complex. In some embodiments, the bispecific molecule referred to herein as T4IL15 (CD4+ T cell targeted IL-15). In some embodiments, T4IL15 is significantly more effective than N803 in activating healthy donor CD4+ memory T cells in vitro. In some embodiments, T4IL15 targets both CD4 and the IL-15 receptor simultaneously.

In some embodiments, the subject matter described herein relates to HIV-1 reactivation in a cohort of PBMC samples obtained from ART-suppressed HIV-1 patients in vitro. Post T4IL15 treatment, it was observed that 7 out of 8 samples tested produced viral RNA, indicative of HIV-1 latent reservoir reactivation. In some embodiments, the subject matter described herein relates to optimizing the effectiveness of latency reactivation by conjugation of HDAC inhibitor to T4IL15. Without being bound by theory delivery of LRAs specifically to resting memory CD4+ T cells and activation through two independent pathways will synergize the efficacy of HIV-1 latency reactivation.

### CrossMab Bispecific Format

In one embodiment, the engineered molecules described herein are in a CrossMab bispecific format. The CrossMab engineering format allows for the bispecific molecule to adopt a more native structure and to assemble correctly two heavy and two light chains, derived from two existing antibodies, to form human bivalent bispecific IgG antibodies. The CrossMab format utilizes the knobs-into-holes technology that enables heterodimerization of two heavy chains. In some embodiments of the CrossMab technology, correct association of the light chains and their cognate heavy chains is achieved by exchange of heavy-chain and light-chain domains within the antigen binding fragment (Fab) of one half of the bispecific antibody. This “crossover” retains the antigen-binding affinity but makes the two arms so different that light-chain mispairing can no longer occur. The creation of a “knob” in one heavy chain and a “hole” in the other heavy chain of the bispecific antibody favors the formation of heavy chain heterodimers, while the “crossover” of CL and CH1 sequences (the constant domains, heavy and light chains) in one arm of the antibody favors correct Heavy-Light chain pairings in both arms. The CrossMab format allows for correct assembly of two heavy chains and two light chains from different parental antibodies into one bispecific antibody molecule that resembles a typical monoclonal antibody in terms of mass and architecture, and with no artificial linkers required. A person of skill in the art would recognize immediately that the CrossMab format can be modified to generate the bispecific molecules disclosed here. More details on the CrossMab format can be found in Schaefer, W., et al, “Immunoglobulin domain crossover as a generic approach for the production of bispecific IgG antibodies” Proc Natl Acad Sci USA. 2011 Jul. 5; 108 (27): 11187-92, which is incorporated herein by references in its entirety.

### T4IL15 Sequences

SEQ ID NO: 1 is iMab-light chain (LC)
DIVMTQSPDSLAVSLGERVTMNCKSSQSLLYSTNQKNYLAWYQQKPGQS
PKLLIYWASTRESGVPDRFSGSGSGTDFTLTISSVQAEDVAVYYCQQYY
SYRTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
SEQ ID NO: 2 is iMab-heavy chain (HC)
(LaLa mutations, LS mutations, hole mutations)
QVQLQQSGPEVVKPGASVKMSCKASGYTFTSYVIHWVRQKPGQGLDWIG
YINPYNDGTDYDEKFKGKATLTSDTSTSTAYMELSSLRSEDTAVYYCAR
EKDNYATGAWFAYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPEFEGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPASIEKTISK
AKGQPREPQVCTLPPSRDELTKNQVSLSCAVKGFYPSDIAVEWESNGQP
ENNYKTTPPVLDSDGSFFLVSKLTVDKSRWQQGNVFSCSVLHEALHSHY
TQKSLSLSPGK

In some embodiments, the LaLa mutations in the sequence encoding the bispecific molecule disclosed herein knock out the Fc function of the bispecific molecule. More information on LaLa Mutations can be found in Lund, J. et al., Human Fc gamma RI and Fc gamma RII interact with distinct but overlapping sites on human IgG, J Immunol, 1991, 15; 147 (8): 2657-62, The contents of which is incorporated herein by reference in its entirety. In some embodiments, the LS mutations in the sequence encoding the bispecific molecule disclosed herein improve the half-life of the bispecific molecule. More information on LS mutations can be found in Zalevsky, J., et al. Nat Biotechnol. 2010 February; 28 (2): 157-9 the content of which is incorporated herein by reference in its entirety. In some embodiments, the hole mutation in one heavy chain and Knob mutations in the other heavy chain are designed to favor heterodimer formation of the two heavy chains. More information on Hole mutations can be found in U.S. Pat. No. 10,308,707, the content of which is incorporated herein by reference in its entirety. Saunders K, Conceptual Approaches to Modulating Antibody Effector Functions and Circulation Half-Life, Front Immunol. 2019, 10:1296 is also incorporated herein by reference in its entirety.

SEQ ID NO: 3 is IL15RaFc (S40C mutation, GSx3 linker,
LaLa mutations, LS mutations, knob mutations)
ITCPPPMSVEHADIWVKSYSLYSRERYICNSGFKRKAGTCSLTECVLNKATNVAHWT
TPSLKCIRDPALVHQRGSGGGGSGGGGSGGGGSDKTHTCPPCPAPEFEGGPSVFLFPP
KPKDTLMISRTPEVTCVVVDVSHEDPEVKFNWYVDGVEVHNAKTKPREEQYNSTYR
VVSVLTVLHQDWLNGKEYKCKVSNKALPASIEKTISKAKGQPREPQVYTLPPCRDEL
TKNQVSLWCLVKGFYPSDIAVEWESNGQPENNYKTTPPVLDSDGSFFLYSKLTVDKS
RWQQGNVFSCSVLHEALHSHYTQKSLSLSPGK

In some embodiments, the S40C mutation forms a disulfide bond with IL-15 mutant polypeptide. In some embodiments, the IL-15 mutant polypeptide has a S52C mutation. In some embodiments, GSx3 is a flexible linker that joins the IL15Rα polypeptide and the IgG Fc polypeptide to form a fusion protein. In some embodiments, the Lala mutations knock out Fc function of the bispecific antibody. In some embodiments, the LS mutations improve the half-life of the bispecific antibody. In some embodiments, the knob mutations are required for bispecific molecule assembly. Lund, J. et al., Human Fc gamma RI and Fc gamma RII interact with distinct but overlapping sites on human IgG, J Immunol, 1991, 15; 147 (8): 2657-62; Zalevsky, J., et al. Nat Biotechnol. 2010 February; 28 (2): 157-9; U.S. Pat. No. 10,308,707; Saunders K, Conceptual Approaches to Modulating Antibody Effector Functions and Circulation Half-Life, Front Immunol. 2019, 10:1296 are all incorporated herein by reference in their entirety.

SEQ ID NO: 4 is IL-15 (L52C) domain
NWVNVISDLKKIEDLIQSMHIDATLYTESDVHPSCKVTAMKCFLLELQVI
SCESGDASIHDTVENLIILANNSLSSNGNVTESGCKECEELEEKNIKEFL
QSFVHIVQMFINTS

### Modified Sequences

SEQ ID NO: 9 is iMab variable light (VL) domain Y32H (Kabat)
DIVMTQSPDSLAVSLGERVTMNCKSSQSLLYSTNQKNHLAWYQQKPGQSPKLLIYW
ASTRESGVPDRESGSGSGTDFTLTISSVQAEDVAVYYCQQYYSYRTFGGGTKLEIK
SEQ ID NO: 10 is IL15 D8A (L52C)
NWVNVISALKKIEDLIQSMHIDATLYTESDVHPSCKVTAMKCFLLELQVISCESGDAS
IHDTVENLIILANNSLSSNGNVTESGCKECEELEEKNIKEFLQSFVHIVQMFINTS
SEQ ID NO: 11 is IL15-GSx3 CH2-CH3 Knob
NWVNVISDLKKIEDLIQSMHIDATLYTESDVHPSCKVTAMKCFLLELQVISLESGDAS
IHDTVENLIILANNSLSSNGNVTESGCKECEELEEKNIKEFLQSFVHIVQMFINTSGSG
GGGSGGGGSGGGGSDKTHTCPPCPAPEFEGGPSVFLFPPKPKDTLMISRTPEVTCVVV
DVSHEDPEVKFNWYVDGVEVHNAKTKPREEQYNSTYRVVSVLTVLHQDWLNGKE
YKCKVSNKALPASIEKTISKAKGQPREPQVYTLPPCRDELTKNQVSLWCLVKGFYPS
DIAVEWESNGQPENNYKTTPPVLDSDGSFFLYSKLTVDKSRWQQGNVFSCSVLHEA
LHSHYTQKSLSLSPGK
SEQ ID NO: 12 is MV1 HC Hole
QVQLQQSGPEVVKPGASVKMSCKASGYTFTSYVIHWVRQKPGQGLDWIGYINPYND
GTDYDEKFKGKATLTSDTSTSTAYMELSSLRSEDTAVYYCAREKDNYATGAWFAY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCD
KTHTCPPCPAPEFEGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPASIEK
TISKAKGQPREPQVCTLPPSRDELTKNQVSLSCAVKGFYPSDIAVEWESNGQPENNY
KTTPPVLDSDGSFFLVSKLTVDKSRWQQGNVFSCSVLHEALHSHYTQKSLSLSPGK
SEQ ID NO: 13 is MV1 LC
DIVMTQSPDSLAVSLGERVTMNCKSSQSLLYSTNQKNYLAWYQQKPGQSPKLLIYW
ASTRESGVPDRFSGSGSGTDFTLTISSVQAEDVAVYYCQQYYSYRTFGGGTKLEIKRT
VAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

### Bispecific Molecule, T41L15, Evaluation

### 1. T4IL15 Expression and Purification

Test agent (TA), also referred to as a bispecific molecule of the present disclosure, was constructed utilizing the knob-into-hole Fc-pairing. Antibody and activator encoding DNA plasmid sequences were cloned into expression vector gWiz and transiently transfected into Expi293 cells at ratio 1:1:1:2 (MVILC:MVIHC:IL15RaFc:IL15). The resulting protein (T4IL15) collected after 6 days of expression was purified by affinity purification using protein A beads. Purification of the T4IL15 protein construct was verified using size exclusion chromatography.

### 2. In Vitro Activity of T4IL15

5×10⁵ PBMCs from twelve healthy donors were stimulated using the test agent (TA) at concentrations of 0.1 μg/ml and 0.02 μg/ml for 18 hours. Following incubation, the cells were washed three times with FACS buffer (PBS+2% FBS), and stained for detection of activation of subset cells using the following cell surface markers shown in Table 1 below:

TABLE 1
Cell Surface Markers.

| Cell surface marker | Antibody | Vendor/Catalog |
|---|---|---|
| CD8 | PerCP-Mouse anti-human CD8 | BD/347314 |
| CD56 | PE-Mouse anti-human CD56 | BD/347747 |
| CD3 | FITC-mouse anti-human CD3 | BD/555332 |
| CD4 | Pacific Blue-Mouse | Biolegend/317429 |
| | anti-human CD4 | |
| CD45R0 | APC/Cy7-Mouse anti- | Biolegend/304228 |
| | human CD45R0 | |
| CD69 | AF700-Mouse anti-human CD69 | Biolegend/310922 |

All staining mAbs were incubated with the PBMCs for 1 hour room temperature following by washing 3 times with FACS buffer. Samples were run on LSRII flow cytometer for analyses of the subsets of cells activated following treatment of test agent.

### 3. Procedure of T Cell Activation Experiment:

Frozen healthy donor PBMCs were thawed, washed to remove cryoprotectant and plated at two million cells per ml of media (20% FCS/RPMI1640) per well in 24-well culture plate. The cells were treated with either (1) 0.1 μg/ml of T4IL15, or (2) a positive control: Dynabeads Human T-cell Activator CD3/CD28 (bead: cell ratio of 1:1) or (3) media only as a negative control in triple replicates. The plates were incubated at 37° C./5% CO₂ for a total of 7 days by replenishing the media on day 3. Cells were harvested on day 1 (18 hours), day 3 and day 7, and stained for flow cytometry analysis with the following cell surface markers shown in Table 2 below.

TABLE 2
Cell Surface Markers

| Cell surface marker | Antibody/Reagent | Vendor/Catalog |
|---|---|---|
| CD45 | PerCP-Mouse anti- | BD/345809 |
| | human CD45 | |
| CD3 | APC-mouse anti-human CD3 | BD/557597 |
| CD4 | FITC-Mouse anti-human CD4 | BD/555346 |
| CD45R0 | APC/Cy7-Mouse anti- | Biolegend/304228 |
| | human CD45R0 | |
| CD69 | AF700-Mouse anti- | Biolegend/310922 |
| | human CD69 | |
| Live/Dead | Fixable Blue Dead Cell Stain | Invitrogen/L23105 |
| | Kit, for UV excitation | |

All antibodies were incubated for 1h before washing with FACS buffer. The samples were run using LSRII flow cytometer and results were analyzed by BD FACSDiva software. The fraction of CD69 positive cells were determined by gating on the CD45RO-positive CD4+CD3+ cells (memory CD4+ T cells) and CD45RO-negative CD4+CD3+ cells (naive CD4+ T cells) at each time points prior to quantitation.

### 4. HIV-1 Viral RNA Quantitation Using RT-PCR:

Viral copy number in the culture supernatant or within the infected cells was measured by the reverse transcriptase-polymerase chain reaction method. Viral RNA was reverse transcribed with Superscript II RT (Invitrogen) to cDNA, which was amplified using AmpliTaq Gold DNA Polymerase (Applied Biosystems). Primers used for amplification include a mixture of the forward primers, RF1+2: (SEQ ID NO: 5) 5′-CGGCGACTGGTGAGTACG-3′ (735-752) and (SEQ ID NO: 6) 5′-GGCGGCTGGTGAGTACG-3′ (736-752), and the reverse primer, RR: (SEQ ID NO: 7) 5′-GACGCTCTCGCACCCAT-3′ (806-790). The tag probe, RB: (SEQ ID NO: 8) 6-FAM-TTTGACTAGCGGAGGCTAGAAGGAGA-BHQ-1 (761-786) (Sigma Genosys, Woodlands, TX), was used to detect the PCR products. A series of 10-fold dilutions of target RNA fragment (532-1419) derived from NL4-3 (0, 1-10⁸ copies) were included in each assay as a standard. Realtime PCR was performed in triplicate using GeneAmp PCR System 9700 (Applied Biosystems) using the following cycling parameters: 95° C., 10 minutes; 50 cycles of 95° C., 15 seconds; and 60° C., 30 seconds. Viral RNA was quantitated as fold increase by assigning the unstimulated control RNA (zero copy) as 1 and using a 5-fold value as a threshold. All samples with a >5-fold increase over the control indicate reactivation of the latent reservoir.

Supernatant from three of the above samples were used to measure production of replication competent virus. Supernatant from the stimulated cultures (from 3) were harvested and examined by using the TZA assay (Gupta, P. et al. 2017 Nature Medicine) and luminescence was measured using Spectramax i3x reader (Molecular Devices).

5. Conjugation Strategy with HDACi and Other Planned Reactivation Molecules to Generate T4IL15-Drug Conjugates (ADC)

In order to improve on our overall activation response, dual-activation strategies for T4IL15 will be employed using an HIV-1 latency reversal agent that has been characterized for clinical use. Table 3 below lists five options based on their mechanism of activation and at least one of them will be used to enhance the reactivation effects of the T4IL15. In some embodiments, an inhibitor of the enzyme histone deacetylase (HDACi) will be conjugated to T4IL15 to generate a dual-activating molecule. The T4IL15 conjugate was generated using the ThioMab technology that genetically engineers a cysteine into the residue A114 of the heavy chain (or the V110A of the light chain) of the ibalizumab arm of the T4IL15. Each of the chosen LRAs will be chemically conjugated to T4IL15 using the chosen bispecific antibodies either directly or through a linker, as shown in Table 3. Both Romidepsin and Disulfiram will be directly conjugated in their reduced form T4IL15 via a disulfide bond (as it has been successfully shown with monoclonal ibalizumab & Romidepsin). To overcome possible bad pharmacokinetics for disulfide linkage in vivo; next generation disulfide re-bridging strategies using bis-maleimide linker to conjugate these payloads forming thioethers and stabilized by Retro-Michael type reaction will be employed, if necessary.

For Ingenols, selective esterification is performed of the primary alcohol in the molecule to the carboxyl group on a heterobifunctional linker also bearing maleimide group and purify the linked payload before conjugating to cysteines on an antibody. If stability of the ester is an issue, we will use a chemoselective oxidation of the alcohols to aldehydes and upon testing the activity of the modified ingenol, we will conjugate the ingenol aldehyde to the antibody via linkers specifically designed for click chemistry. For acitretin, the carboxyl group will be connected to amine group on a heterobifunctional linker using well-established carbodiimide chemistry and the purified adduct linked by maleimide-thiol linkage to the chosen bispecific antibody.

Following conjugation, the ADC will be carefully analyzed to ensure that the structure and iMab and IL15 epitope specificities have not been adversely altered. In addition, LC-MS analysis will be conducted to measure the conjugation efficiency and measure an appropriate drug: antibody ratio. Once the ADCs are properly quality controlled, they will be studied for the targeting effect of the vehicle or the functional effect of the payload, or both. Potential molecules for conjugation with T4IL15 are provided in Table 3 below.

TABLE 3
Molecules for Conjugation with T4IL15

| | | | In vitro Functional |
|---|---|---|---|
| LRA class | Example | Conjugation Method | Assay for payload |
| HDAC | Romidepsin | Disulfide/maleimide linker | Acetylation of histones |
| inhibitor | | using thioether bond | on target cells |
| PKC | Ingenol | Ester bond to carboxylate | Phosphorylation of synthetic |
| activator | mebutate | group on bifunctional linker | substrate by cell lystates with |
| | | | activated protein kinase |
| PTEN | Disulfiram | Disulfide/maleimide linker | PTEN activity in cell lysate |
| inhibitor | | using thioether bond | |
| RIG-1 | Acitretin | Amide bond on bifunctional | p300 levels measuring |
| Activator | | linker | HAT activity |
| SMAC | Birinapant, | Amide bond to bifunctional | Luminescent caspase activation |
| mimetic | | linker | assay |

### Pharmaceutical Compositions

In some embodiments, the subject matter disclosed herein provides a pharmaceutical composition comprising any of the bispecific molecules disclosed herein. In some embodiments, the pharmaceutical composition disclosed herein further comprises one or more pharmaceutically-acceptable diluents, one or more pharmaceutically-acceptable carriers, or one or more pharmaceutically-acceptable excipients.

In some embodiments, the subject matter disclosed herein provides one or more polynucleotides encoding the any of the bispecific molecules disclosed herein. In some embodiments, the subject matter disclosed herein provides one or more genetically engineered cells comprising any of the bispecific molecules disclosed herein. In some embodiments, the subject matter disclosed herein provides one or more genetically engineered cells comprising a polynucleotide encoding the any of the bispecific molecules disclosed herein.

In certain aspects, provided herein are pharmaceutical compositions comprising the above-described bispecific molecules (or one or more polynucleotides encoding one or more bispecific molecules). In some embodiments, the subject matter described herein relates to a pharmaceutical composition comprising an effective amount of the bispecific molecules (or one or more polynucleotides encoding one or more bispecific molecules) described herein and a pharmaceutically-acceptable diluent, carrier or excipient. In certain embodiments, the bispecific molecules are conjugated with therapeutic molecules to increase the effectiveness the bispecific molecules disclosed herein, as is known by those practiced in the art. In some embodiments, the therapeutic molecules are HDAC inhibitors.

As used herein, “pharmaceutical composition” refers to a therapeutically effective formulation according to the invention. A “therapeutically effective amount,” or “effective amount,” or “therapeutically effective,” as used herein, refers to an amount which provides a therapeutic effect for a given condition and administration regimen. In some embodiments, the condition is an HIV infection. In some embodiments, the condition is an early stage of an HIV infection. In some embodiments, the condition is a late stage of the HIV infection. In some embodiments, the condition is AIDS. A therapeutically effective amount can be determined by a skilled person based on patient characteristics, such as age, weight, sex, HIV viral load, complications, other diseases the subject is suffering from, etc., as is well known in the art.

In some embodiments, the pharmaceutical compositions described herein can be administered as solid compositions. In some embodiments, the solid compositions comprise one or more excipients including, but not limited to, lactose, starch, cellulose, milk sugar or high molecular weight polyethylene glycols. In some embodiments, the pharmaceutical compositions described herein can be administered as aqueous suspensions and/or elixirs. In some embodiments, the pharmaceutical compositions described herein may be combined with various sweetening or flavoring agents, coloring agents or dyes, emulsifying agents, suspending agents and with diluents such as water, ethanol, propylene glycol and glycerin, and combinations thereof.

In some embodiments, the pharmaceutical compositions described herein can be administered parenterally, for example, intravenously, intra-arterially, intraperitoneally, intra-thecally, intraventricularly, intrasternally, intracranially, intra-muscularly or subcutaneously, or they may be administered by infusion techniques. In some embodiments, the pharmaceutical compositions described herein can be administered in the form of a sterile aqueous solution which may contain other substances, for example, enough salts or glucose to make the solution isotonic with blood. The preparation of suitable parenteral formulations under sterile conditions is readily accomplished by standard pharmaceutical techniques well-known to those skilled in the art.

In some embodiments, pharmaceutical compositions suitable for parenteral administration include, but are not limited to, aqueous and non-aqueous sterile injection solutions which may contain anti-oxidants, buffers, bacteriostats and solutes which render the formulation isotonic with the blood of the intended recipient. Further such compositions include aqueous and non-aqueous sterile suspensions which may also include suspending agents and thickening agents. The pharmaceutical compositions described herein can be presented in unit-dose or multi-dose containers. The pharmaceutical compositions can be scaled containers, ampoules or vials. The pharmaceutical compositions can be stored in a freeze-dried (lyophilized) condition requiring only the addition of the sterile liquid carrier, such as water for injections, immediately prior to use.

The compounds and pharmaceutical compositions of the present invention can be employed in combination therapies, that is, the bispecific molecules and pharmaceutical compositions can be administered concurrently with, prior to, or subsequent to, one or more other desired therapeutics or medical procedures (e.g., bispecific molecules can be used in combination treatment with another treatment such as antibodies). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic). For the purposes of an example the another therapeutic of prophylactic can be one of more antiretroviral drugs, which functions by stopping the HIV from replicating in the body.

The invention also provides a pharmaceutical pack or kit comprising one or more containers filled with one or more of the ingredients of the pharmaceutical compositions of the invention. In some embodiments, the container(s) can come with a notice in the form prescribed by a governmental agency regulating the manufacture, use or sale of pharmaceuticals or biological products. The notice can reflect approval by the agency of manufacture, use or sale for human administration.

The compositions described herein can be formulated with appropriate carriers and adjuvants using techniques to yield compositions suitable for prophylaxis or treatment. In some embodiments, the adjuvant can be alum, poly IC, MF-59, squalene-based adjuvants, or liposomal based adjuvants suitable for prophylaxis or treatment.

### Production of the Bispecific Molecules

In some embodiments, the bispecific molecules disclosed herein are be produced by any method known in the art. In some embodiments, the bispecific molecules disclosed herein are produced by culturing a cell transfected or transformed with a vector comprising one or more nucleic acid sequences encoding the bispecific molecules described herein. In some embodiments, the methods disclosed herein include expressing the bispecific molecules of the disclosure and then isolating those bispecific molecules.

In some embodiments, bispecific molecules are synthesized by methods which results in bispecific molecules that are not contaminated by immunoglobulins. In some embodiments, the bispecific molecules of the present disclosure may be made by a variety of techniques known in the art, including, for example, the hybridoma method, recombinant DNA methods, phage-display technologies, B-cell discovery methods, and technologies for producing human or human-like antibodies in animals that have parts or all of the human immunoglobulin loci or genes encoding human immunoglobulin sequences.

In some embodiments, expression of a bispecific molecule comprises expression vector(s) containing one or more polynucleotides that encodes a bispecific molecule. Methods that are well known to those skilled in the art can be used to construct expression vectors comprising bispecific molecule coding sequences and appropriate transcriptional and translational control signals. These methods include, for example, in vitro recombinant DNA techniques, synthetic techniques, and in vivo genetic recombination. Particular embodiments provide replicable vectors comprising one or more nucleotide sequence encoding a bispecific molecule disclosed herein operably linked to a promoter. In preferred embodiments, such vectors may include a nucleotide sequence encoding the heavy chain of a bispecific molecule (or fragment thereof), a nucleotide sequence encoding the light chain of a bispecific molecule (or fragment thereof), or a nucleotide sequence encoding both the heavy and light chain of a bispecific molecule (or fragment thereof).

The one or more polynucleotides encoding the bispecific molecules may be modified, for example, by substituting the coding sequence for human heavy- and light-chain constant domains or by covalently joining to the immunoglobulin coding sequence all or part of the coding sequence for a non-immunoglobulin polypeptide. Such non-immunoglobulin polypeptides can be substituted for the constant domains of bispecific molecules.

Various expression systems for producing bispecific molecules are known in the art, and include, prokaryotic (e.g., bacteria), plant, insect, yeast, and mammalian expression systems. Suitable cell lines, can be transformed, transduced, or transfected with nucleic acids containing coding sequences for bispecific molecules or portions of the bispecific molecules disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes.

A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

In some embodiments, the bispecific molecules disclosed herein are encoded in one or more vectors for expression in a cell line. In some embodiments, one or more vectors comprises one or more polynucleotide sequence that encode bispecific molecules and the vector is transfected into one or more cell lines for expression. In some embodiments, one or more vectors comprise polynucleotide sequences encoding a light chain, a heavy chain, or any other chain of interest of the bispecific molecules. For example, in some embodiments, a first vector may comprise a polynucleotide sequence encoding a light chain, a second vector may comprise a polynucleotide sequence encoding a heavy chain, of a bispecific molecule. In some embodiments, a vector may comprise a polynucleotide sequence encoding any domain of a bispecific molecule. In some embodiments, the necessary vectors are transfected into one or more cell lines for expression of the bispecific molecule. A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign bispecific molecule expressed. Thus, eukaryotic host cells possessing the cellular machinery for proper processing of the primary transcript, glycosylation, and phosphorylation of the gene product may be used.

Various culturing conditions and methodologies can be used with respect to the host cells being cultured. Appropriate culture conditions for mammalian cells are well known in the art or can be determined by the skilled artisan (see, for example, Animal Cell Culture: A Practical Approach 2^{nd} Ed., Rickwood, D. and Hames, B. D., eds. (Oxford University Press: New York, 1992)). A person of skill would understand that cell culturing conditions can vary according to the type of host cell selected and/or commercially available media can be utilized.

The bispecific molecules disclosed herein can be purified from any human or non-human cell that expresses the bispecific molecules, including those that have been transfected with one or more expression constructs that express the bispecific molecules. For recovery, isolation and/or purification of the bispecific molecules, the cell culture medium or cell lysate is centrifuged to remove particulate cells and cell debris. The desired bispecific molecule can be isolated or purified from contaminating soluble proteins and polypeptides by suitable purification techniques. Non-limiting purification methods for proteins/antibodies include: size exclusion chromatography, affinity chromatography, ion exchange chromatography, ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during bispecific molecules purification. Purification procedures that can select for carbohydrates can also be used, e.g., ion-exchange soft gel chromatography, or HPLC using cation- or anion-exchange resins, in which the more acidic fraction(s) is/are collected.

### Methods of Treatment

In some embodiments, the subject matter disclosed herein relates to a preventive medical treatment started after following diagnosis of a disease (e.g., HIV infection) in order to prevent the disease from progressing. In one embodiment, the subject matter disclosed herein relates to prophylaxis of subjects who are believed to be at risk for moderate or severe disease associated with HIV infection. In some embodiments, the subjects can be administered the pharmaceutical composition described herein comprising one or more bispecific molecules described herein. It is contemplated using any of the bispecific molecules produced by the systems and methods described herein. In some embodiments, the compositions described herein can be administered subcutaneously via syringe or any other suitable method known in the art.

The bispecific molecules disclosed herein or the pharmaceutical compositions may be administered to a cell, mammal, or human by any suitable means. In some embodiments, one or more bispecific molecules disclosed herein are prepared in a cocktail of DNA or mRNA sequences encoding the bispecific molecules described herein and delivered to a subject for in vivo expression of the encoded bispecific molecules.

As will be readily apparent to one skilled in the art, the effective in vivo dose to be administered and the particular mode of administration will vary depending upon the age, weight and species treated, and the specific use for which the compound or combination of compounds disclosed herein are employed. In some embodiments, the determination of effective dose levels (i.e., the dose levels necessary to achieve the desired result) is accomplished using routine pharmacological methods. In some embodiments, human clinical applications of products are commenced at lower dose levels, with dose level being increased until the desired effect is achieved. In some embodiments, acceptable in vitro studies can be used to establish useful doses and routes of administration of the compositions identified by the present methods using established pharmacological methods. Effective animal doses from in vivo studies can be translated to appropriate human doses using conversion methods known in the art.

In certain aspects, the subject matter disclosed herein provides a method of treating or preventing an HIV infection in a subject in need thereof, the method comprising administering to the subject any of the bispecific molecules disclosed herein or any pharmaceutical compositions thereof. In some embodiments, the bispecific molecules disclosed herein activate CD4+ T-cells. In some embodiments, the bispecific molecules reduce activation of CD8+ T cells. In some embodiments, the bispecific molecules described herein activate cells expressing both IL-2 receptor and CD4. In some embodiments, the bispecific molecules described herein reverse transcriptional silencing of HIV expression.

In certain aspects, the subject matter disclosed herein provides a method of reactivating a HIV-1 reservoir in a subject in need thereof in a subject in need thereof, the method comprising administering to the subject any of bispecific molecules disclosed herein or any pharmaceutical compositions thereof. In some embodiments, the bispecific molecules disclosed herein activate CD4+ T-cells. In some embodiments, the bispecific molecules reduce activation of CD8+ T cells. In some embodiments, the bispecific molecules described herein activate cells expressing both IL-2 receptor and CD4. In some embodiments, the bispecific molecules described herein reverse transcriptional silencing of HIV expression.

In some embodiments, in the methods disclosed herein the subject is a human subject.

### Kits of the Invention

In one embodiment, the subject matter disclosed herein relates to a kit for generating the bispecific molecules comprising the bispecific molecule composition of the present invention and instructions for use. In one embodiment, the subject matter disclosed herein relates to a kit for generating the bispecific molecules comprising one or more vectors comprising one or more polynucleotide sequence encoding any of the bispecific molecules described above. The kit can further include at least one additional reagent or one or more of the bispecific molecules of the present invention. The kit usually has a label indicating the intended use of the kit contents. The term label includes all documents and is attached to the kit or with the kit, or otherwise attached to the kit.

### EXAMPLES

The following examples illustrate the present invention, and are set forth to aid in the understanding of the invention, and should not be construed to limit in any way the scope of the invention as defined in the statements of the invention which follow thereafter. The Examples described below are provided to illustrate aspects of the present invention and are not included for the purpose of limiting the invention.

### Example 1—Evaluating Strategies for HIV-1 Latency Reversal Ex Vivo and In Vivo

In some embodiments, the subject matter disclosed herein relates to reversing HIV-1 latency using any of the bispecific molecules described herein. In some embodiments, a latent HIV-1 reservoir is established during primary infection. In some embodiments, the latent HIV-1 reservoir comprises a group of immune cells in the body of the infected subject. In some embodiments, these immune cells are infected with HIV-1 but are not actively producing new HIV particle. In some embodiments, the immune cells are CD4+ T cells. In some embodiments, the reservoir comprises primarily HIV-1-infected and long-lived subpopulations of CD4+ resting memory T cells. In some embodiments, the latent HIV-1 reservoir is established during the second or third stages of the HIV infection. In some embodiments, the subject matter described herein relates to methods of targeting and/or eliminating the latent HIV-1 reservoirs.

While currently available antiretroviral therapies (ART) can reduce the level of HIV in blood to an undetectable level, they cannot eliminate the latent reservoir, thereby imposing a major obstacle to curing the infection. Therefore, developing strategies to eliminate or to reduce the viral reservoir that could lead to a cure or lifelong remission of HIV-1 infection remains a key priority in HIV/AIDS research. Among strategies being pursued toward eliminating the latent reservoirs, the “Shock-and-Kill” approach, aims to induce HIV-1 expression from latent infected cells using latency reversal agents (LRAs). HIV reactivation could facilitate the clearance of these cells either by viral cytopathic effect, or by host immune responses, with the ultimate goal of reducing the size of the viral reservoirs. A number of small molecule LRAs, such as histone deacetylase (HDAC) inhibitors and Protein Kinase C (PKC) activators, have shown viral reactivation in vitro in generating viral transcripts or virus-like particles, but they have little or no impact on the reduction of latent reservoirs in physiologically relevant concentration in clinical trials. Increasing the dose of LRAs is prohibitive in their current forms due to the potential systemic toxicity. IL-15 is an inflammatory cytokine produced by antigen-presenting cells and induces selective activation and proliferation of natural killer (NK) cells and T cells. IL-15 has also been shown to activate HIV in infected memory CD4+ T cells bearing IL-15 receptor by producing viral gene transcripts in vitro. Due to its potential in activating the latent reservoir while enhancing the effector function of host immune responses, one recent study on phase I clinical trial to assess the safety and virologic impact of the IL-15 super-agonist N-803 (IL-15RaFc) in people living with ART suppressed HIV was reported (Miller J S et al 2022). This study showed that 1) N-803 was safe and well-tolerated at the tested doses, 2) the administration of N-803 was associated with proliferation and activation of CD4+, CD8+ T cells, and NK cells, and 3) a modest reduction in the inducible HIV reservoir was observed in PBMCs from participants receiving the agonist. Our lab has been exploring targeted delivering of LRAs using exquisite specificity of monoclonal antibodies to CD4+ T cells. We hypothesize that 1) a bispecific molecule targeting both CD4 and IL-15 receptor simultaneously will be selective to CD4+ T cells and more effective than untargeted IL15RaFc in specific activation of the resting memory CD4+ T cell population, and 2) chemical conjugation of HDAC inhibitor to this CD4-targeted molecule will further synergize latency reversal efficiency.

Evaluating T4IL15 and T4IL15-Remidepsin conjugation for HIV latency reversal ex vivo using PBMC samples from ART suppressed persons living with a HIV infection.

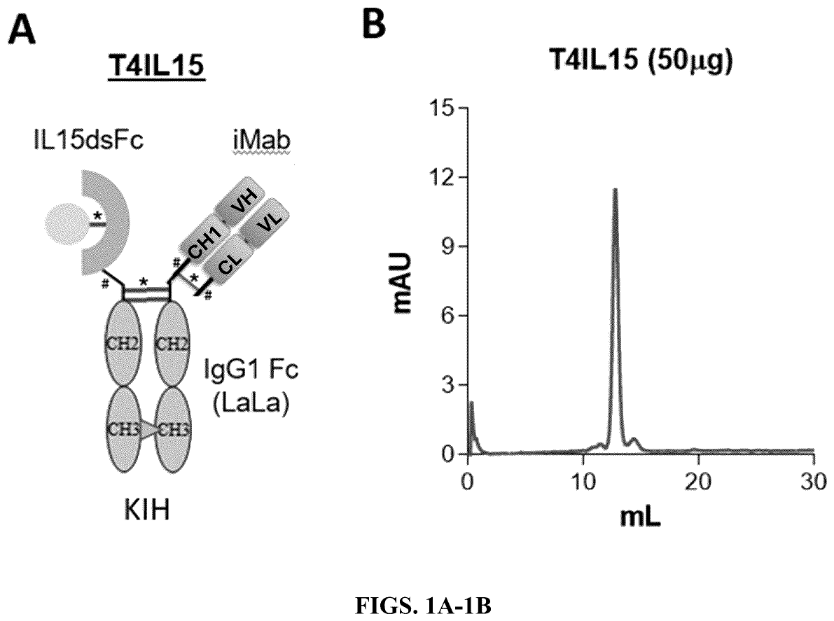

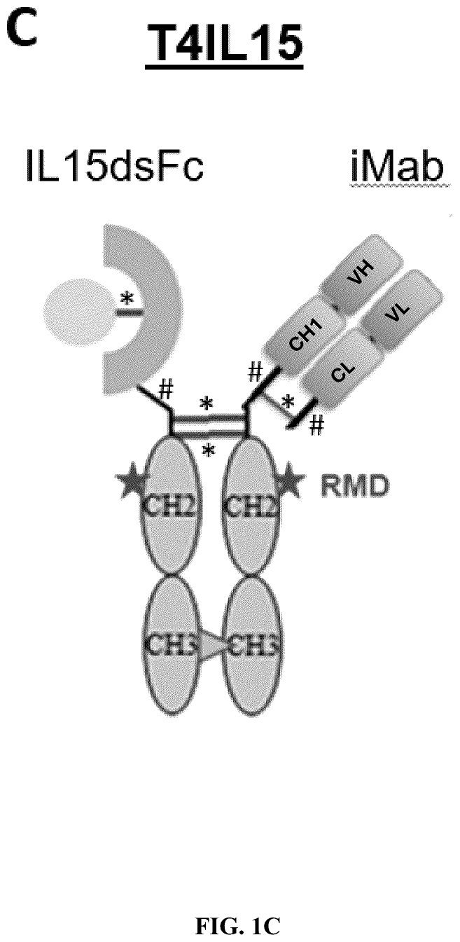

In some embodiments, the subject matter described herein relates to the construction of a bispecific molecule with one arm containing anti-CD4 mAb iMab (Ibalizumab, FDA approved HIV drug) and other arm containing IL-15 or IL-15 and Sushi domain of receptor α chain complex IL-15Rα. In some embodiments, the bispecific molecules are capable of binding IL-12/IL-15 receptor IL-2Rβ (CD122) and common gamma chain (γC, CD132). In some embodiments, the bispecific molecules are capable of binding CD4. In some embodiments, the bispecific molecules disclosed herein comprise a first and a second arms wherein the first arm has a first antigen binding region that binds CD4 on the surface of a CD4+ T cell and the second arm has a second antigen binding region that binds IL-15 or IL-15 and Sushi domain of receptor α chain complex IL-15Rα. In some embodiments, the bispecific molecule is referred to as T4IL15 (CD4+ T cell targeted IL-15, FIGS. 1A-C). In some embodiments, the bispecific molecule is referred to as T4IL15(-Rα).

FIG. 1A shows a schematic representation of a T4IL15 bispecific molecule design. The schematic shows a bispecific molecule with an IL15dsFc arm, which contains the IL15 and Sushi domain complex. FIG. 1B shows a Size Exclusion Chromatography (SEC) profile for T4IL15, a chromatographic method in which molecules in solution are separated by their size and/or molecular weight. FIG. 1C shows a schematic of another representation of the bispecific molecules described herein. This is a construct design where the molecule is conjugated to the HDAC inhibitor Romidepsin (RMD). The schematic shows a bispecific molecule with an IL15dsFc arm, which contains the IL15 and Sushi domain complex.

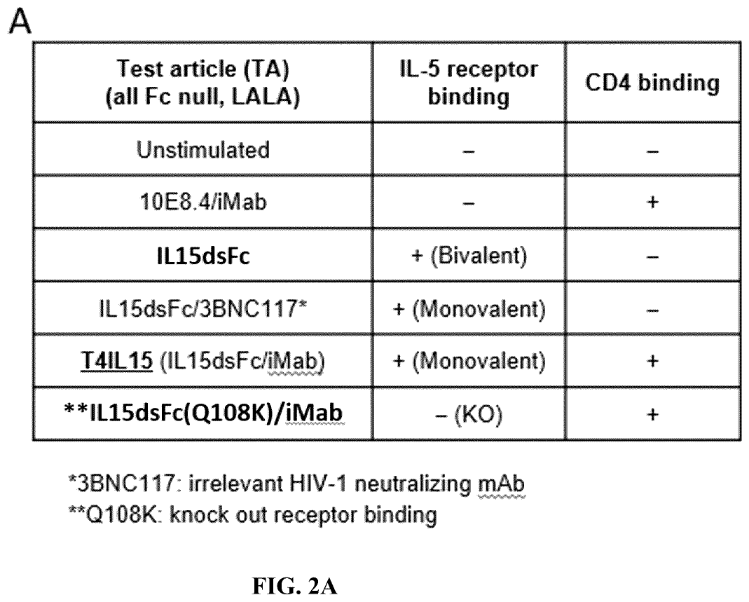

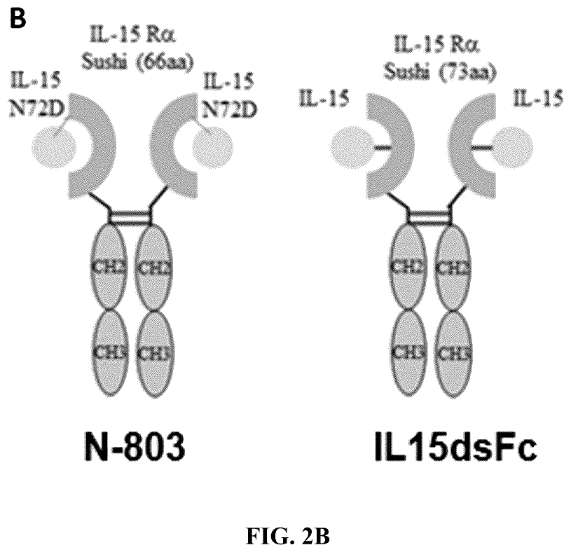

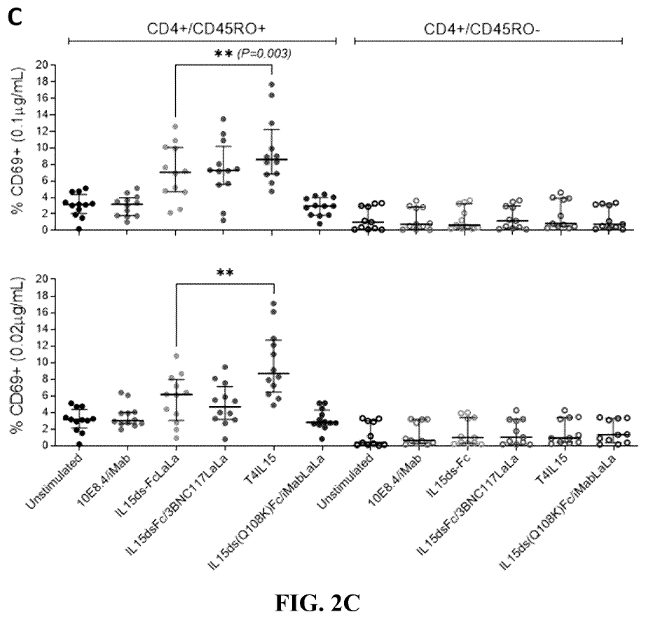

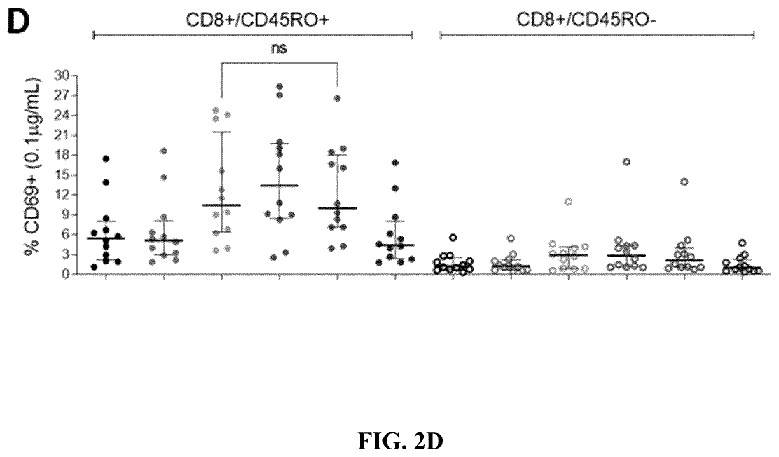

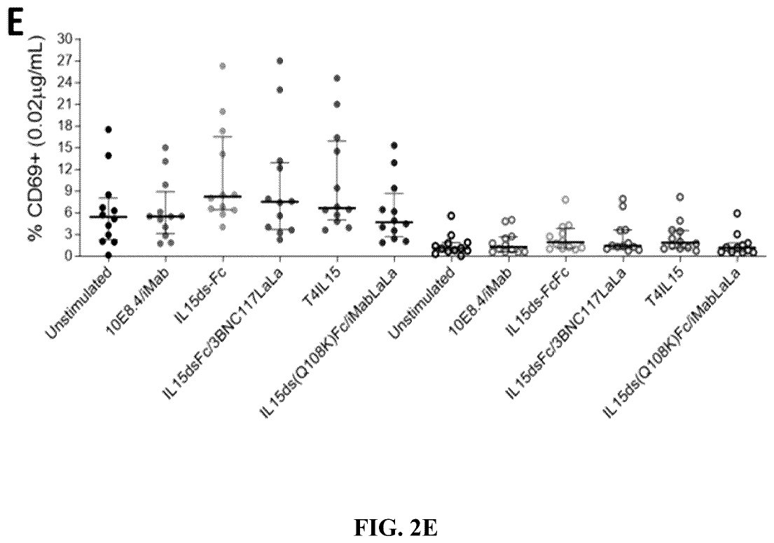

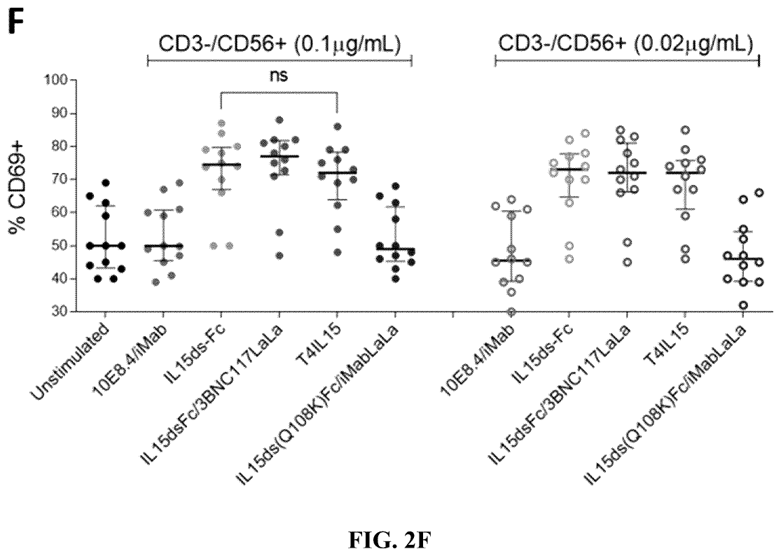

The stimulative activity of T4IL15 on CD69 expression was examined using healthy donor PBMCs. As shown in FIGS. 2A-D, T4IL15 was significantly more effective than untargeted IL-15RaFc in activating healthy donor CD4+ memory, but not naive T cells in vitro, and enhanced potency likely through bispecific binding of to both CD4 and IL15 receptors. FIG. 2A shows stimulation of PBMCs from each donor with respective test articles. 5×10⁵ PBMCs from each donor (150 μL, n=12) were stimulated with the respective test articles (TAs) at concentrations of 0.1 μg/mL or 0.02 μg/mL. Stimulation lasted for approximately 18 hours.

FIG. 2B shows the design of test articles N-803 and IL15dsFc, both of which are monoclonal molecules. In the design of N-803, the Sushi domain of IL15Rα consists of 66 amino acids and there is a N72D mutation in the sequence encoding IL-15 (IL-15N72D). In some embodiments, the N72D mutation enhances IL-15 binding to IL-2Rβ (CD122). In the design of IL15dsFc (P22339), the Sushi domain of IL15Rα consists of 73 amino acids, there is a S40C mutation in the sequence encoding the IL15Rα, and a L52C mutation in the sequence encoding IL-15. In some embodiments, the S40C and the L52C mutations are introduced to form a disulfide bond between IL15Rα and IL-15. FIG. 2C shows CD69 upregulation of CD4+ T cells measured ˜18 hours following stimulation. T4IL15 activates CD45RO+ memory CD4+ T cells, but not CD45RO-naïve CD4+ T cells with 0.1 μg/ml or 0.002 μg/ml of TA. FIGS. 2D-E show that IL15dsFc/iMab activates CD45RO+ memory CD8+ T cells, but not CD45RO-naïve CD8+ T cells with 0.1 μg/ml (FIG. 2D) or 0.002 μg/ml (FIG. 2E). IL15dsFc/iMab and IL15ds-Fc have a similar potency in activating CD8+ memory T cells in vitro. Activation of CD8+ T cells is likely through IL-15 trans-presentation. FIG. 2F shows that T4IL15 retains the ability to activate NK cells. T4IL15 and IL15ds-Fc have a similar potency on NK cell activation in vitro. Activation NK cells by T4IL15 is likely through IL-15 trans-presentation

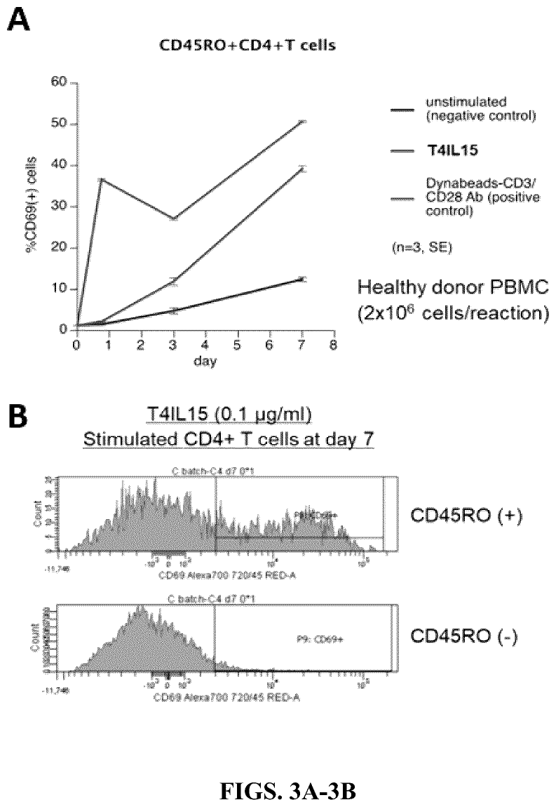

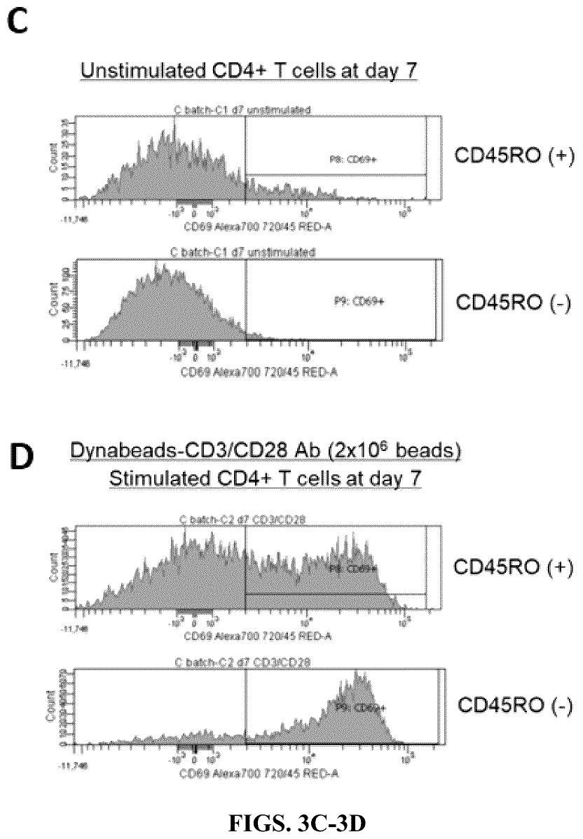

CD69 is a marker for early T cell activation signaling through the NF-κB pathway, which correlates with HIV-1 latency reversal. IL-15 activates CD45RO+ memory CD4+ T cells, but not CD45RO-naïve CD4+ T cells. T4IL15 has an enhanced potency of CD4+/CD45RO+ T cell activation as compared to the bivalent IL15ds-Fc, likely through bispecific binding of both CD4 and IL15 receptors. CD4+ memory T cell activation depends on the IL-15 receptor binding, but not on the CD4 receptor binding. In a time-course analysis, even at day 7 post stimulation, CD69 expression was selectively detected in CD4+memory but not CD4+naïve T cells (FIGS. 3A-D). FIG. 3A shows an increase in CD69+ cells with T4IL15 stimulation over 7 days. The CD45RO antigen, an isoform of CD45 antigen, is a marker of memory T cells, which proliferate in response to recall antigen. By the expression of the CD45RO antigen, CD4+ T cells are sub-grouped into CD45RO-positive memory CD4+ T cells and CD45RO-negative naive CD4+ T cells. FIG. 3B indicates the histogram for the CD69 signal and shows the fraction of CD69 positive cells for CD45RO+CD4+ T cells and CD45RO-CD4+ T cells at day 7 following stimulation with T4IL15. Similarly, FIG. 3C shows the fraction of CD69 positive cells for CD45RO+CD4+ T cells and CD45RO-CD4+ T cells at day 7 without stimulation (negative control). FIG. 3D shows the fraction of CD69 positive cells for CD45RO+CD4+ T cells and CD45RO-CD4+ T cells at 7 days following stimulation by the Dynabeads-CD3/CD28 Antibody (positive control).

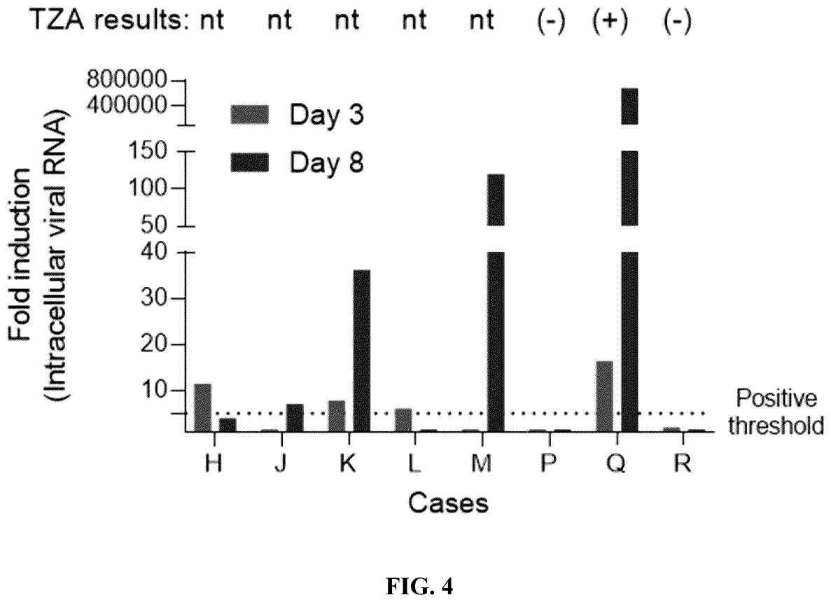

In a pilot study, HIV-1 reactivation was assessed in a cohort of PBMC samples obtained from ART-suppressed HIV-1 patients in vitro. Post T4IL15 treatment, 6 out of 8 samples tested were positive for intracellular viral RNA production, indicative of HIV latent reservoir reactivation (FIG. 4). To measure the production of replication competent virus, the latency reversal analysis was repeated using 3 available PBMC samples. 7-day post T4IL15 treatment, culture supernatants were harvested and tested for virus outgrowth using TZA analysis (Sanyal, A et al. 2017). Among these, only samples showing HIV RNA production post stimulation were TZA positive (FIG. 4), providing evidence of both latency reactivation and production of replication competent virus. In the future this selective reactivation of T4IL15 will be enhanced by conjugating HDACi Romidepsin (RMD) to T4IL15 (FIG. 1C). Using site-specific conjugation, studies linking RMD to iMab showed that this conjugate has a remarkable selectivity and RMD-mediated histone acetylation was an order of magnitude higher in CD4+ cells when treated with the conjugate than when treated with free RMD (data not shown). Therefore, without being bound by theory, delivery of LRAs, such as IL-15 and RMD, specifically to resting memory CD4+ T cells and activation through two independent pathways will synergize the efficacy of HIV latency reactivation. A series of analyses of physicochemical properties such as homogeneity, conjugation efficiency, and stability will be conducted to ensure the good quality of the products. Upon procurement and validation of the essential reagents, a head-to-head comparative study will also be conducted to evaluate the efficacy of latency reversal. In brief, CD4+ T cell will be purified from PBMC and stimulated with IL-15RaFc, T4IL15 and T4IL15-RMD. Intracellular HIV RNA will be quantified by RT-PCR at day 3 and day 7 post stimulation, and culture supernatant will also be harvested at the same time point for detection of replication competent virus by TZA.

### Example 2—Evaluating the Efficacy of T4IL15-RMD on Latency Reactivation in HIV Infected Humanized Mice Fully Suppressed with ART

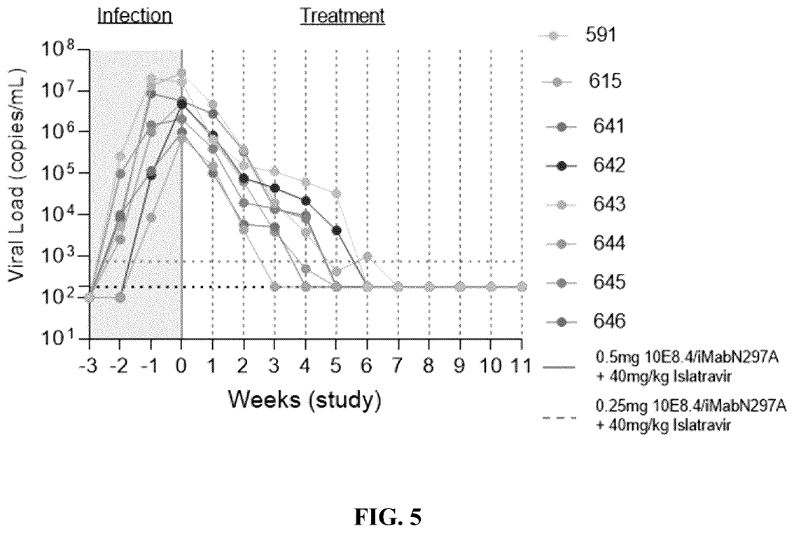

Both, SHIV/SIV infection of macaques and HIV infection of immunodeficient mice engrafted with human T cells, have been found to exhibit viral dynamics similar to those found in infected humans and can be used as animal models of HIV infection. Likewise, the establishment of a latent reservoir early in the course of infection has been shown in both models. However, ibalizumab is a humanized antibody and IL-15 is human origin, both of which will likely be immunogenic in macaques. This complication limits monkey studies on the proposed strategy to short-term experiments, which are not suitable when evaluating the long-lived latent reservoir. This then leaves the humanized mouse model of HIV infection, which is not saddled with immunogenicity issues since the mice are immunodeficient. To be able to achieve full viral suppression in humanized mice, the higher dosing of antiviral treatment is required and daily injections of the ART often generate injection site lesions in mice making then infeasible for prolonged use. To overcome these concerns, a regimen combining long-acting ARV with potent neutralizing antibody would be more tolerable and feasible. A combination regimen of the highly potent bispecific antibody 10E8.4/iMab (Huang, Y. et al., 2016) and a potent second-generation reverse transcriptase inhibitor Islatravir was identified with intracellular half-life of days (Stoddart, C. et al., 2015). A weekly regimen of 40 mg/kg of the drug in combination with 20 mg/kg of 10E8.4/iMab initial dose followed by 10 mg/kg of later doses achieved full suppression of virus for 6 continuous weeks of treatment in 8 humanized mice that were infected with HIV-1 AD8 virus (FIG. 5). At the end of the study, all humanized mice with full virus suppression maintained normal peripheral CD4 T cell counts and CD4+ T cell subset phenotype profiles were similar to human PBMCs (data not shown). The sustained viral suppression seen in this case is likely due to potential synergy effect of the combination of bispecific antibody with Islatravir.

### Example 3—T4IL15 with Modified Sequences

It was previously shown, in ART-treated macaques infected with SIV, that N-803 treatment alone does not reactivate SIV virus production. After CD8+ T cell depletion, N-803 treatment induced robust and persistent reactivation of the virus. Co-culture with CD8+ T cells blocked the in vitro latency-reversing effect of N-803 on primary human CD4+ T cells that were latently infected with HIV. Activation of memory CD8+ T cells was found to (1) induce transcriptional silencing of HIV expression, (2) stabilize the HIV reservoirs, and (3) contribute to HIV persistence. More details on the effects of N-803 on reactivation are described in Nature Vol 578 6-February 2020, “Robust and persistent reactivation of SIV and HIV by N-803 and depletion of CD8+ cells,” which is incorporated herein by reference in its entirety.

In some embodiments, the subject matter described herein relates to improved CD4+ T cell targeting design of the IL-15 arm of T4IL15. In some embodiments, the subject matter described herein relates to mutating the IL-15 arm of T4IL15. In some embodiments, the IL-15 mutation is a L52C substitution. In some embodiments, the mutation is a D8A substitution. In some embodiments, the computation comprises a L52C substitution and a D8A substitution. In some embodiment, the subject matter described herein relates to reducing memory CD8+ T cell activation. In some embodiments, the reduced memory CD8+ T cell activation is achieved through mutating the IL-15 arm of T4IL15. In some embodiments, the IL-15 mutation is a L52C substitution. In some embodiments, the mutation is a D8A substitution. In some embodiments, the computation comprises a L52C substitution and a D8A substitution.

Without being bound by theory, reducing IL15 binding affinity to IL-2 receptor β/γ [T4IL15(D8A)] can decrease memory CD8+ T cell activation and improve the selectivity for CD4+ T cell activation.

In some embodiments, the subject matter described herein relates designing one or more mutations to reduce the binding affinity of the anti-CD4 arm of T4IL15 (the iMab arm). Without being bound by theory, lowered binding affinities to both the IL-2 receptor and CD4 can improve the binding avidity of T4IL15 to the targeted cells exclusively expressing both receptors. T4ΔIL15(D8A) can further reduce memory CD8+ T cell activation while retaining the memory CD4+ T cell activation thus improving selectivity.

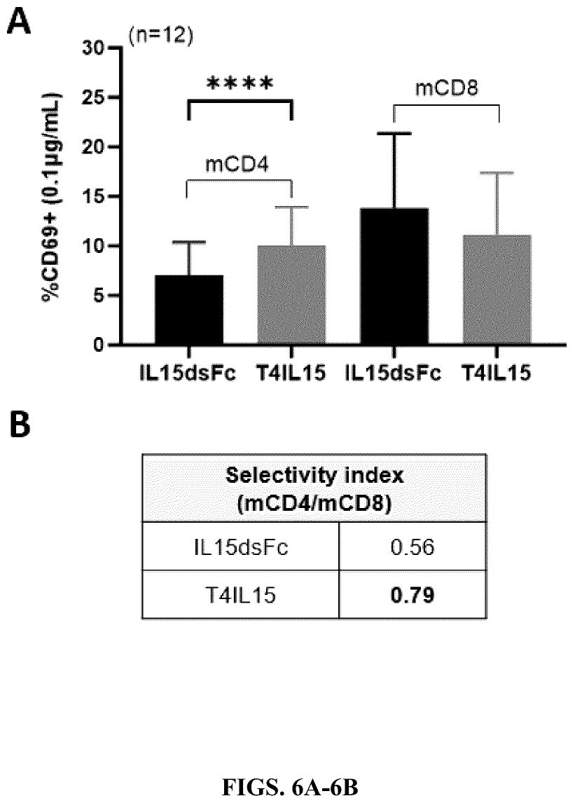

FIGS. 6A-B show selectivity of T4IL15 for CD4. To improve the specificity of memory CD4+ T cell activation, IL15dsFc and T4IL15 activation data from FIGS. 2C and 2D were extracted, replotted and compared (FIG. 6A). Memory CD4+ T cell (mCD4) and CD8+ T cell (mCD8) activations were compared specifically for each stimulator. FIG. 6A shows that a statistically significant improvement of mCD4 T cell activation was achieved by T4IL15. FIG. 6B shows that IL15dsFc has a selectivity index of 0.56, whereas T4IL15 has an index of 0.79, suggesting that T4IL15 selectivity induces mCD4+ T cells over mCD8+ T cells. The ratio of mCD4 over mCD8 is calculated as a selectivity index.

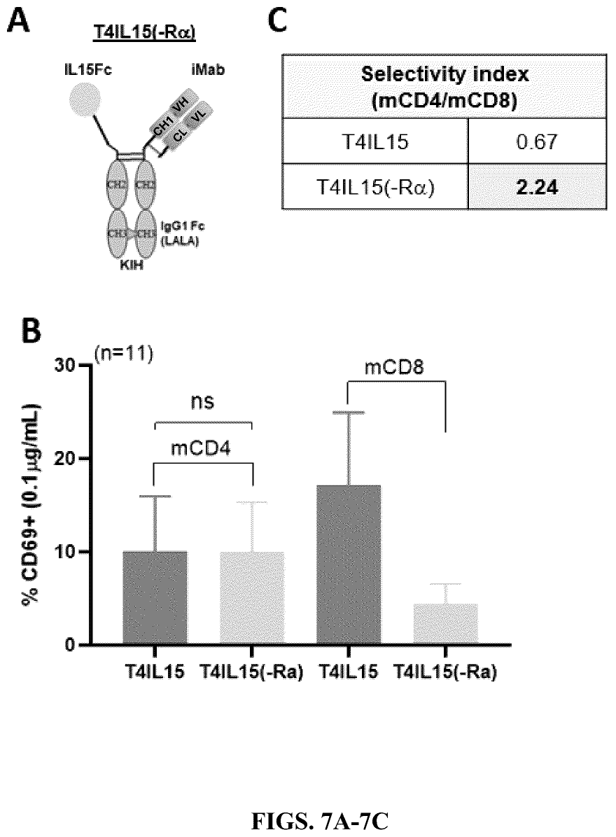

FIGS. 7A-C show IL15 design modifications to enhance selectivity. FIG. 7A shows T4IL15 bispecific construct without an IL15 and Sushi domain receptor α complex (called T4IL15(-Rα)). FIG. 7B shows T4IL15(-Rα) activation of CD4+ T cells. FIG. 7C shows reduced memory CD8+ T cell activation by T4IL15(-Rα). Without being bound by theory, since activation in mCD8+ by T4IL15 is likely due to IL15's high-affinity trans-presentation to mCD8, the reduction in binding affinity of IL15 to its receptor will reduce memory CD8+ T cell activation. It was previously shown that IL15 without receptor α (a component of the IL15 receptor complex) reduced binding affinity to IL-2 receptor β and γ complex by about 100-fold. A T4IL15 bispecific construct was designed without IL15 receptor α (called T4IL15(-Rα) (FIG. 7A). T4IL15(-Rα) comprises IL15-GSx3 CH2-CH3 knob (SEQ ID NO: 11), MV1 HC hole (SEQ ID NO: 12), and MV1 LC (SEQ ID NO: 13). Compared to parental T4IL15, T4IL15(-Rα) retained memory CD4+ T cell activation (FIG. 7B) but reduced memory CD8+ T cell activation with a selective index of 2.24 (FIG. 7C). This data supports the suggestion that reducing IL15 binding can indeed improve the intended selectivity.

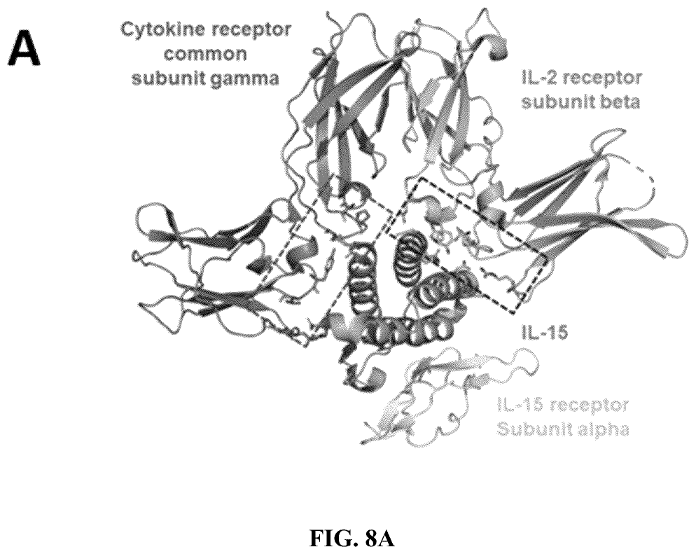

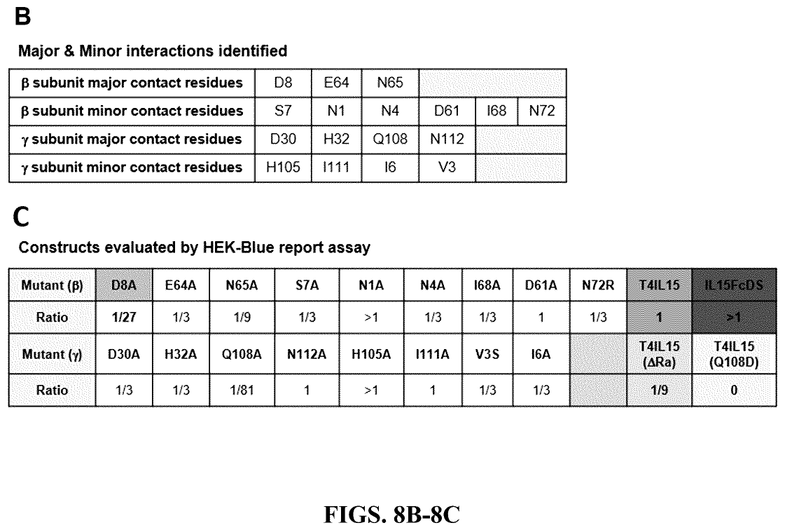

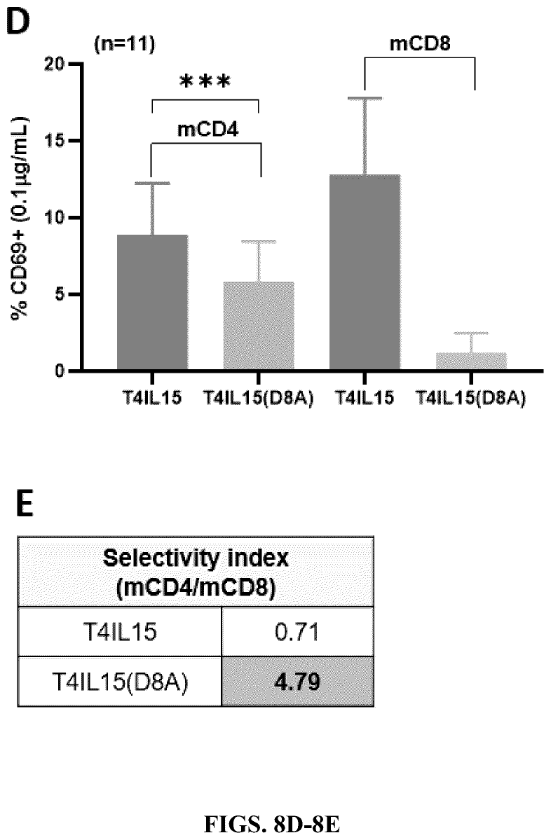

FIGS. 8A-E show that IL15 mutant with reduced binding affinity improves selectivity. FIG. 8A shows in silico modeling of interactions between IL15 cytokine with IL2 receptor β and common chain γ. FIG. 8B shows identification of these interactions. FIG. 8C shows constructs evaluated by HEK-Blue Report assay. FIG. 8D shows activation of memory CD4+ T cells or memory CD8+ T cells in human PBMC-based CD69 upregulation following candidate administration. FIG. 8E shows improved selectivity index for T4IL15(D8A). Based on in silico modeling (FIG. 8A), the major and minor interactions of amino acid residues on IL15 cytokine with IL2 receptor β and common chain β were identified (FIG. 8B). As listed in FIG. 8C, these residues were replaced with alanine on the T4IL15 backbone. IL-2 and IL-15 reporter cells (HEK-Blue, www.invivogen.com/hek-blue-il2) were first used to screen for the low-affinity binding mutants to candidate IL15. Activity of T4IL15 was used as a benchmark to derive the activity ratio. As indicated, the candidates with an activity ratio lower than T4IL15ΔRα were further tested for activation of memory CD4 or memory CD8 cells in human PBMC-based CD69 upregulation (FIG. 8D). T4IL15(D8A) was down-selected as the candidate from the tested panel of mutants since this mutant, while reducing memory CD4+ T cell activation in comparison to original T4IL15, improved the selectivity index significantly (FIG. 8E).

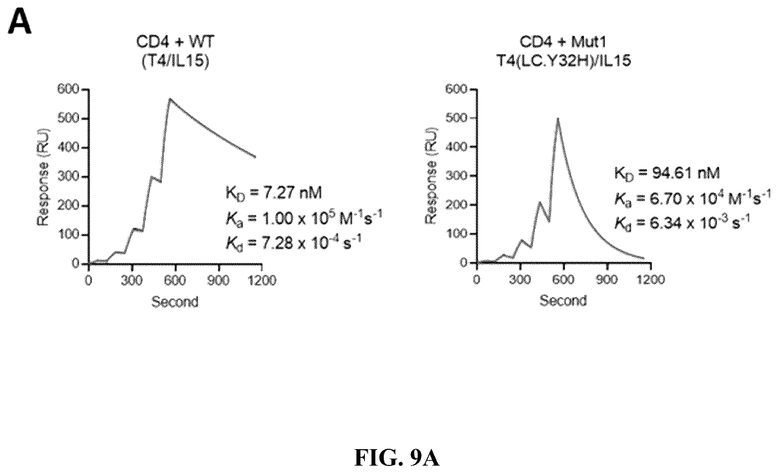

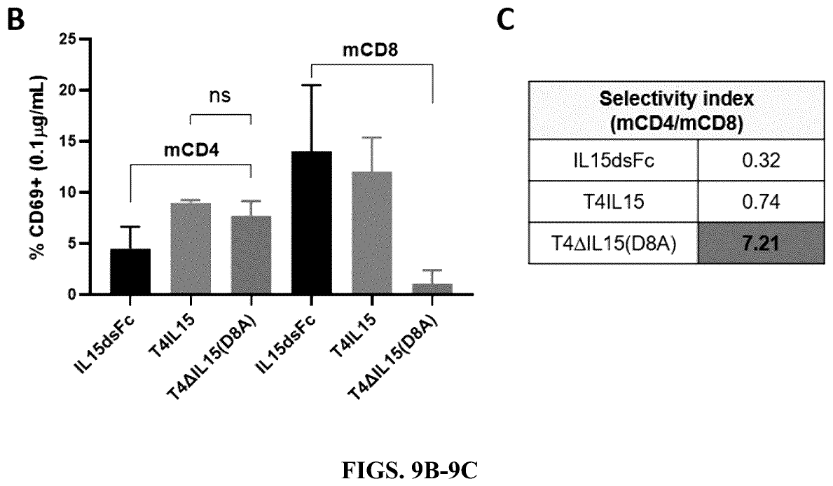

FIGS. 9A-C show modifications to the CD4 binding arm of the bispecific molecules described here to further enhance selectivity. FIG. 9A shows reduced binding affinity to CD4 by T4Δ(LC.Y32H)IL15 construct as compared to parental T4IL15 in surface plasmon resonance (SPR) binding analysis. FIG. 9B shows T4ΔIL15(D8A) retained memory CD4+ T cell activation at comparable levels to parental T4IL15. FIG. 9C shows T4ΔIL15(D8A) reduced memory CD8+ T cell activation, thereby increasing the selectivity index to 7.21. Previously, a light chain CDR1 mutation was identified at position Y32H of the anti-CD4 monoclonal iMab antibody that demonstrated reduction of CD4 binding on the surface of CD4 T-cells by approximately 10-fold. Consistent with this cell surface-based binding data, the T4Δ(LC.Y32H)IL15 construct reduced binding affinity to CD4 by ˜13-fold as compared to parental T4IL15 in surface plasmon resonance (SPR) binding analysis (FIG. 9A). In PBMC based CD69 upregulation analysis (FIG. 9B), T4ΔIL15(D8A) retained memory CD4+ T cell activation at comparable levels to parental T4IL15. Importantly, T4ΔIL15(D8A) reduced memory CD8+ T cell activation, thereby increasing the selectivity index to 7.21 (FIG. 9C). Thus far, the optimization efforts described herein have significantly improved selectivity index from 0.79 of T4IL15 (FIG. 6B), to 4.79 of T4IL15(D8A) (FIG. 8E) to 7.21 of T4ΔIL15(D8A) (FIG. 9C). This improved construct can be used to lead to more effective HIV latency reactivation in subjects in need thereof.

### Example 4 Ex Vivo Reactivation of HIV from Patient PBMC with Controlled Viremia

The engineered bispecific T4ΔIL15(D8A) was compared to the original constructs T4IL15 and IL15RadsFc for their ability to reactivate latent infectious HIV-1 from antiretroviral therapy (ART) treated people living with HIV (PLWH) with no measurable virus in the blood. An ex vivo reactivation strategy was employed where the patient-derived peripheral blood monocytes (PBMC) from the PLWH were applied to measure HIV-1 latency reversal.

PBMC from patients with controlled viremia on suppressive ART were treated directly with each of the embodiments and comparators (IL15RadsFc, T4IL15 and T4ΔIL15(D8A)) and an irrelevant CD4-targeting bispecific antibody at 0.1 ug/mL dose for a period of 5 days before the supernatant was collected and centrifuged for viral particle isolation. Phorbol myristoyl acetate (50 ng/ml) combined with 1 μM ionomycin (PMA/Io) was used as a positive control for the virus reactivation assay based on previously published studies. After 5 days of incubation, viral particles were pelleted from the supernatant for RNA extraction. RNA was quantified from each of the sample treatments by performing a one-step reverse transcriptase-polymerase chain reaction (RT-PCR) that amplified the Pol gene in the virus, and the levels of the RNA were quantified by a standard curve of the Pol gene from HIV-1 virions with known infectivity titers.

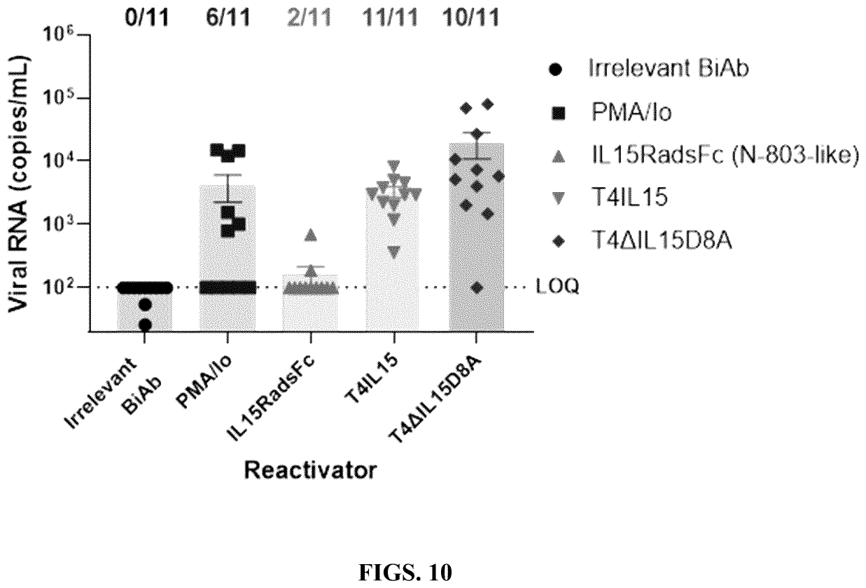

Quantifiable HIV-1 production was noted in PBMCs reactivated with the embodiments at 5 days of culturing. Data from the cohort of PBMC samples indicate successful detection of viral RNA, as depicted in FIG. 10. Both the original (T4IL15) and optimized (T4ΔIL15(D8A)) targeted constructs outperformed the untargeted comparator (IL15RadsFc/N803-equivalent) and the positive control (PMA/Io) in the frequency and magnitude of viral reactivation in the 11 samples assessed. The engineered bispecifics displayed the production of viral RNA in 90-100% samples as compared to positive control (PMA/Io) that showed a reactivation frequency in only 55% of the PBMCs tested. The magnitude of viral particles (copies/ml of viral RNA) produced by the targeted embodiments were generally comparable or higher than PMA/Io.

### Example 5—In Vivo Reactivation of HIV-1 by Novel Optimized T4IL15 in Humanized Mouse Models

Despite the profound success of combination antiretroviral therapy (ART) in suppressing plasma viremia to undetectable levels, HIV-1 persists in long-lived latently infected resting CD4+ T cells, forming a stable reservoir that is the principal barrier to cure. Current latency-reversing agents (LRAs) have demonstrated only modest efficacy in vivo and are often limited by non-specific immune activation or systemic toxicity. IL-15-based immunotherapeutics, such as the IL-15 superagonist N803, have shown the capacity to induce HIV reactivation in vitro and augment antiviral immune responses in vivo. However, their clinical use has been constrained by potent activation of CD8+ T cells and associated toxicities. To overcome these limitations, T4IL15, a novel IL-15-based construct, and its engineered derivative T4ΔIL15(D8A), in which the D8A mutation reduces IL-2 receptor binding and global IL-15 mediated activity while preserving or enhancing CD4+ T cell responsiveness, were developed. The objective of this in vivo study was to evaluate the ability of T4IL15 and T4ΔIL15D8A to reactivate latent virus in HIV-infected humanized NSG (hu-NSG) mice that were otherwise aviremic from treatment with suppressive ART. Modeling viral reactivation potential in vivo aimed to establish the selectivity, efficacy, and translational potential of the T4IL15-based strategy for latency reversal and thereby HIV cure strategies.

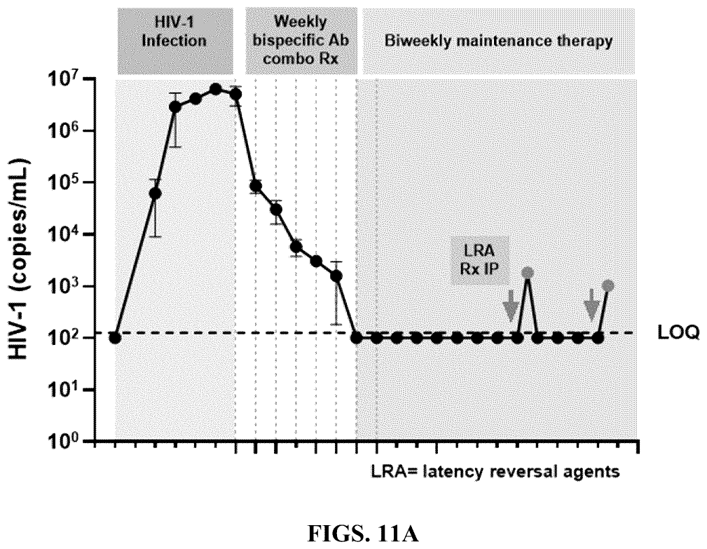

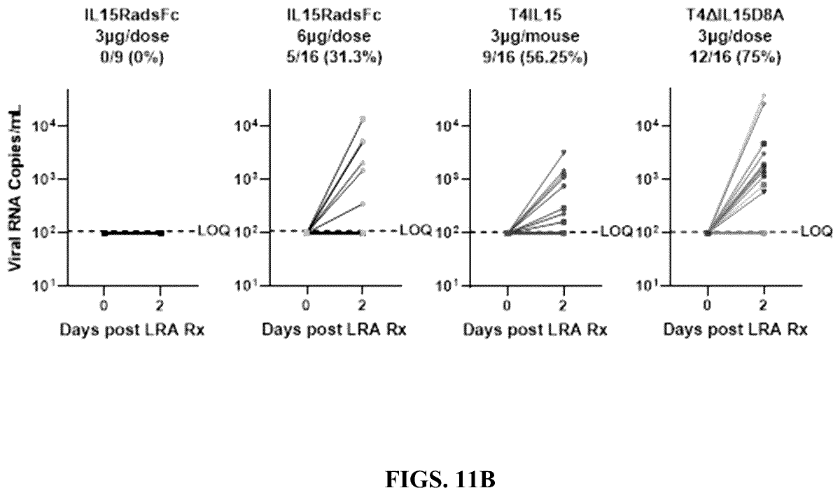

NSG mice were reconstituted with human hematopoietic stem cells and subsequently infected with HIV-1 strain AD8 at a dose of 30,000 TCID50 as performed in FIG. 5. The humanization status of mice was monitored by quantification of circulating human CD4+ T cell counts, as adequate reconstitution is a prerequisite for HIV infection, persistence, and reactivation. Following steady-state infection, the mice were treated with a cocktail of bispecific antibodies (10E8.4/iMab 297A and PGT121/VRC07-LS) on a weekly basis to achieve complete suppression of active viral replication. Once complete suppression was achieved, the mice were kept on maintenance therapy throughout the remaining period of experimentation as depicted in FIG. 11A. To ensure full suppression prior to experimental intervention, mice were required to demonstrate at least two consecutive undetectable plasma viral load measurements. On the day of initiation of the reactivation, mice were first bled for checking absence of viremia and presence of human CD4 to levels greater than the threshold. Post bleeding, study was initiated by injecting mice with indicated doses: (3/6 μg) of IL15RadsFc or 3 μg of T4IL15 or 3 μg of T4ΔIL15(D8A). A total of 9 doses were tested for IL15RadsFc (3 μg) and 16 doses of each IL15RadsFc (6 μg), T4IL15 (3 μg) and T4ΔIL15(D8A) (3 μg). Two days post each dosing; mice were bled for extracting plasma RNA to measure levels of HIV-1 using one-step RT-PCR that amplified the levels of the Pol gene in the blood and served as a marker to indicate viral reactivation. Viral blips are quantitated as copies/mL based on a standard curve run in parallel to the samples.

The copies/mL of viral RNA from the cohort of aviremic mice subjected to each of the latency reactivating embodiments were combined to calculate the cumulative performance of each test agent. As shown in FIG. 11B, the untargeted IL15RadsFc showed measurable viral blips in ART-suppressed hu-NSG mice only in 31.3% of the 2-fold higher (6 ug) doses and in 0% of the 3 ug doses provided to mice. In contrast, the targeted T4IL15 when given at 3 μg dose was able to reactivate virus in 56.25% of the recipient mice and the T4ΔIL15(D8A) at the same dose performed better with 75% of the recipient mice having a reactivation event by day 2 of the dosing indicating that the optimized version was more targeted and selective to inducing reactivation of the virus from resting CD4+ reservoir cells in vivo in our humanized mouse model.

1. A bispecific molecule comprising:
a first arm comprising a first polypeptide and a second polypeptide, wherein the first polypeptide comprises a heavy chain and the second polypeptide comprises a light chain, wherein the heavy chain is coupled to the light chain,
a second arm comprising a third polypeptide, wherein the third polypeptide comprises a CH2 domain and aCH3 domain of a heavy chain, wherein the second arm further comprises an IL-15 domain; and
wherein the heavy chain of the first arm and the CH2-CH3 domains of the second arm form a knob-into-hole structure.

2. The bispecific molecule of claim 1, wherein the first arm forms a first antigen binding region, wherein the first arm binds CD4 with the first antigen binding region, and/or the second arm forms a second antigen binding region, wherein the second arm binds an IL2/IL-15 receptor β/γ heterodimer with the second antigen binding region.

3-13. (canceled)

14. The bispecific molecule of claim 1, wherein the second arm of the bispecific molecule further comprises a Sushi domain of an IL-15 receptor α chain.

15-17. (canceled)

18. The bispecific molecule of claim 1, wherein;
a) the light chain of the first arm comprises SEQ ID NO: 1 or an amino acid sequence with at least 80% identity to SEQ ID NO: 1; or
the light chain of the first arm comprises a variable (VL) domain and a constant (CL) domain, wherein the VL domain comprises SEQ ID NO: 9 or an amino acid sequence with at least 80% identity to SEQ ID NO: 9;
b) the heavy chain of the first arm comprises SEQ ID NO: 2 or an amino acid sequence with at least 80% identity to SEQ ID NO: 2;
c) the second arm comprises SEQ ID NO: 3 or an amino acid sequence with at least 80% identity to SEQ ID NO: 3; or
the second arm comprises SEQ ID NO: 11 or an amino acid sequence with at least 80% identity to SEQ ID NO: 11; and/or
d) the IL-15 domain comprises SEQ ID NO: 4 or an amino acid sequence with at least 80% identity to SEQ ID NO: 4; or
the IL-15 domain comprises SEQ ID NO: 10 or an amino acid sequence with at least 80% identity to SEQ ID NO: 10.

19-29. (canceled)

30. The bispecific molecule of claim 1, wherein the bispecific molecule is conjugated to a histone deacetylases (HDAC) inhibitor, a PKC activator, a PTEN inhibitor, a RIG-1 activator, or a SMAC mimetic.

31-39. (canceled)

40. The bispecific molecule of claim 1, wherein the bispecific molecule is T4IL15, T4IL15(-Rα), or T4ΔIL15(D8A).

41. (canceled)

42. A pharmaceutical composition comprising the bispecific molecule of claim 1.

43. A polynucleotide encoding the bispecific molecule of claim 1 or a fragment thereof.

44. A virus or genetically engineered cell comprising a polynucleotide of claim 43.

45. A genetically engineered cell comprising a bispecific molecule of claim 1.

46-88. (canceled)

89. A method of treating latent HIV-1 infection or reactivating a HIV-1 reservoir in a subject in need thereof, the method comprising administering to the subject a bispecific molecule comprising:
a first arm comprising a first polypeptide and a second polypeptide, wherein the first polypeptide comprises a heavy chain and the second polypeptide comprises a light chain, wherein the heavy chain is coupled to the light chain,
a second arm comprising a third polypeptide, wherein the third polypeptide comprises CH2-CH3 domains of a heavy chain, wherein the second arm further comprises an IL-15 domain; and
wherein the heavy chain domain of the first arm and the CH2-CH3 domains of the second arm form a knob-into-hole structure.

90. The method of claim 89, wherein the first arm forms a first antigen binding region, wherein the first arm binds CD4 with the first antigen binding region, and/or the second arm forms a second antigen binding region, wherein the second arm binds an IL2/IL-15 receptor β/γ heterodimer with the second antigen binding region.

91-99. (canceled)

100. The method of claim 89, wherein the second arm of the bispecific molecule further comprises a Sushi domain of an IL-15 receptor α chain.

101-103. (canceled)

104. The method of claim 89, wherein;
a) the light chain of the first arm comprises an amino acid sequence with at least 80% identity to SEQ ID NO: 1; or
the light chain of the first arm comprises a variable (VL) domain and a constant (CL) domain, wherein the VL comprises SEQ ID NO: 9 or an amino acid sequence with at least 80% identity to SEQ ID NO: 9;
b) the heavy chain of the first arm comprises SEQ ID NO: 2 or an amino acid sequence with at least 80% identity to SEQ ID NO: 2;
c) the second arm comprises SEQ ID NO: 3 or an amino acid sequence with at least 80% identity to SEQ ID NO: 3; or
the second arm comprises SEQ ID NO: 11 or an amino acid sequence with at least 80% identity to SEQ ID NO: 11; and/or
d) the IL-15 domain comprises SEQ ID NO: 4 or an amino acid sequence with at least 80% identity to SEQ ID NO: 4; or
the IL-15 domain comprises SEQ ID NO: 10 or an amino acid sequence with at least 80% identity to SEQ ID NO: 10.

105-115. (canceled)

116. The method of claim 89, wherein the bispecific molecule is conjugated to a histone deacetylases (HDAC) inhibitor, a PKC activator, a PTEN inhibitor, a RIG-1 activator, or a SMAC mimetic.

117-125. (canceled)

126. The method of claim 89, wherein the bispecific molecule is T4IL15, T4IL15(-Rα), or T4ΔIL15(D8A).

127-130. (canceled)

131. The method of claim 89, wherein the reservoir is a latent reservoir, wherein the latent reservoir comprises HIV-1 infected CD4+ T cells.

132. (canceled)

133. A bispecific molecule means for binding IL15 Receptor and CD4.

134. The means of claim 133, wherein the means comprises the bispecific molecule of claim 1.

135. The means of claim 133, wherein the means is capable of treating a HIV-1 infection in a subject in need thereof or reactivating a HIV-1 reservoir in a subject in need thereof.


SEQUENCE LISTING (13 sequences)

SEQ ID NO: 1 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGK

SEQ ID NO: 3 - protein, 317 aa, synthetic construct
ITCPPPMSVEHADIWVKSYSLYSRERYICNSGFKRKAGTCSLTECVLNKATNVAHWTTPSLKCIRDPALVHQRGSGGGGSGGGGSGGGGSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 4 - protein, 114 aa, synthetic construct
NWVNVISDLKKIEDLIQSMHIDATLYTESDVHPSCKVTAMKCFLLELQVISCESGDASIHDTVENLIILANNSLSSNGNVTESGCKECEELEEKNIKEFLQSFVHIVQMFINTS

SEQ ID NO: 5 - DNA, 18 bp, synthetic construct
  source 1..18: mol_type = other DNA
cggcgactggtgagtacg

SEQ ID NO: 6 - DNA, 17 bp, synthetic construct
  source 1..17: mol_type = other DNA
ggcggctggtgagtacg

SEQ ID NO: 7 - DNA, 17 bp, synthetic construct
  source 1..17: mol_type = other DNA
gacgctctcgcacccat

SEQ ID NO: 8 - DNA, 26 bp, synthetic construct
  source 1..26: mol_type = other DNA
  modified_base 1: mod_base = OTHER; note = 6-Carboxyfluorescein modified nucleotide
  modified_base 26: mod_base = OTHER; note = Black hole quencher 1 modified nucleotide
tttgactagcggaggctagaaggaga

SEQ ID NO: 9 - protein, 112 aa, synthetic construct
DIVMTQSPDSLAVSLGERVTMNCKSSQSLLYSTNQKNHLAWYQQKPGQSPKLLIYWASTRESGVPDRFSGSGSGTDFTLTISSVQAEDVAVYYCQQYYSYRTFGGGTKLEIK

SEQ ID NO: 10 - protein, 114 aa, synthetic construct
NWVNVISALKKIEDLIQSMHIDATLYTESDVHPSCKVTAMKCFLLELQVISCESGDASIHDTVENLIILANNSLSSNGNVTESGCKECEELEEKNIKEFLQSFVHIVQMFINTS

SEQ ID NO: 11 - protein, 358 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGGGSGGGGSGGGGSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 12 - protein, 452 aa, synthetic construct; identical to SEQ ID NO: 2

SEQ ID NO: 13 - protein, 219 aa, synthetic construct; identical to SEQ ID NO: 1